SEQUENCE LISTING (20 sequences)

SEQ ID NO: 1 - DNA, 3556 bp, Mycovirus MoCV3
  CDS (121)..(3501)
gcaaaaaagagaataaagctttctcctttttgcactccattcaccggttaaaatcgcacgatcagcccgcacaattaagccattattagaaaaagctcactcaacgcagaagcttcgacgatggcaggtggttatgatgaggatgtaggctcactggggtcaggtgagccagtgttcggcgcatcatacacgaacatctggcactcaacgctgctggcggctgaggggtcagtggctcacagtgggcctgcgctgtacgcaatcgtgttgcccctcagttgtgggaagtccagcttagcctccgtgctcagcgggtacgatattgatgatatggtggttaactcggcggccttacatgccgacgatgaatggcgcacgatgctggatgcgcgatcgaaaggttgggcgtacgaggacaaggcggcttaccggctggcaaacgacctgatgctccgtcgggctaggaggttcctccgcgcgttcgaaggcgacgataacgcccctgttgtgtacgtgcacacgcgggaactcgcaactgctttggggctgcggatcatattcgatgggtacgtagaggaggctgcgtggctgggttgtcgccgccagttggaatccgacgcagtgacacgcgatcgcgacttgcgagcgtatcgtggccaggtagctgccaaccgggcgcacgcgatacgccacaggcaacctgagccagtcccgtacacatcgcactcccggttggctgaggcggccgaagccgctatcacccgcgctggcctatgcgctgggagcccgcgtgacttggcggacaggactaagttatgcggggcaccaccgcaaattatgcttgacttggcgcactcgatctgccgtgatcgccaccggccagcctggttgcgggcggtggctgccaagctgctgcgataccggatgggcgaggtgttgccacaggaggcgctggcggccgacaactatagtgagtgggcgagggtgatccacgcgactgaccagcaccgtgtggctgaggctcccgcacaatcgctgagagggcagaactggtcagaggtttttccgtatggtgcgggcaactcgcgatttgctttagttaagatcggtgattggatcgattgcacaggcacgagtgcgatgggcttcggttacgagtggttccgccagatggtgaccagacgagagggcacgtatgaacaggcgagctgcatgctgctgatgggcgatgtgttcgactacatggcccccgagttgcacccgctgatccaacgcttacccatggggtcactgaggttggagcattatgctgagatcgcgaaggagattcacaggctagtgcgctcgagcgtaacgttgctgggccgccgcctggacgcaggccagctctccgtgtgcacctactgggattgcttagcggggcgctatttgggctcgggcgacatggagaaggagctggctgaccggaccagcgagcagaaaccgcgcgtgtgggtgtcacgggatgggacgcagtcagccgaccggttcgcgcatgagttcgcttgtgaggtcagggctttactgcaccaaacaatagctgacggtggtgagcaaatgcgcaccgtcacggacatggtggcgtcattcgacacgttcctagagtatcgcaaaaagtgggtgagaccggggtccgtaacaggcagccccaaggccgatatatatttggaggccgtgagcgagagagaggggatgattgccgaagtcgctgacgacattgcggcgatgggcacgtacgtactggctaacgtccggctgaataaggctgccacgtttgagttcgctgaattcccggccatagtaaagcgcgtgctggcggactatgtgccgaacagcttcacaaggtacttcataaagaacgagattggcaagccagcgggccgagcgctttacccgtcccacctggcgcattacgtagcggggcagttcgcactgtacgccctgatgaaggcgcaacctatccctaaggtgcgcttggcctcagaacgcgacgtggctatggatgaacactggatgtggatgcaggcccgtgagttcaccgtgggtgtgatgctggattacgacaacttcaacgaaaagcatgaattcgcggatatgcaacttatcatgcgggagctgaaaggattgtatcgcacagcgggcgtattgagccccgatttaaagaccatgatagattgggtggcggaggcgtatgaccgcacggttttggagtacgatggcgaactgcacagtttcaagcacggcatgctatccgggcaggcgcctacctccgcaataaacaacataataaacggtgcgaacaaaaggttgttgataaggcaggtggaggagctaacaggccgggtgatattccagaagcgcacgtctggcggcgacgacgtggccggcgagacctactcactgtacgacgcatacctagctgtgaagtgcgggcagcagatggggctggccttcaaggatataaaacagctcttaagctccgattactatgaattttttcggctatttgtgagtgtgaagggcgtgcacgggtcgctgcctcgtgcgttgggtagcatctgctcaggccagtggtcgaacagcgtcaaagccaaattcgtcgacccggcggccaagctatcgtccgtgactgatgcggccttcaagatagctcgccgggcggggggcaacgccacattccgcgagaagctatgcgccacagcgttcaagaaatgggcttcatacaacgagcaggcgctagtcagggggtttatccacggtgaaaggcactccggcggcctcggtgtcccgatgagtgacggctccgtgctacgcatagagccaatccagtggccagatgaggagagggtgcgtctcaaaggcttgcctaaggacgcgagccaagtggtggtggaagatgccgtgaaacaagcgaccgagttagtagggcctgatagcgtggagtcggccgaagttgttgccaaccggctgagtgagcaggtgttcaaagcgaacgtggcagcgatggaagggtcgcgggtcggtcagctactcggatcatgggaggggccgcgtcatgtcagggtgcacgaagtgctgcgtatctcggaggccgacgtggccgcaacggcgcccaccgcagaagaattcagggcggcgtacgccaagcacaagacgataatagaatactaccgcaaagcaggggctaagtatgatgcgttagccggggtagtcaagcccaaggcgcgtgaaaagctggcgcgcgcgtcatgcaacggcacgccgtgtgactacaagaaactgtacttctggaaagagcatttaacaatgtacggctgtggaacgtacctgctgactgaagacacttacgatgcggcaagtatgctggcgctagtggtgagttcagagctgtcgaatgaagcggtgagtcgtcggttggctgaatgcgcggtcgcactgaagcgcgccgggctggtgagctactgatcgggcaggtggcacacagcacaaaaacttcttacgctgtgaggtgtaagta

SEQ ID NO: 2 - DNA, 3252 bp, Mycovirus MoCV3
  CDS (341)..(3142)
aaaaaagagaataaagcattcccttttttgcactttgcgcgacctactcagatggcagctcaaccgaaccgtggcttcgactcattcttcgcagattactacactcaagacggcttttcggacctaaccagctcacggccatctaccatccgcccagccaacatagtggtggaaccagaccaggaagtcttcgacgcctaccgcgcacacggcgggagccctccggactacacaaccagtcaagtagctgacgcgttcggccagcgtgcttcggcgagtaacagaatccacggagtcaggcgagacacagccgcctctgtacacctcgcaggacgaagcgatgttgatgcgcgagagcaggggtatggaagaggaggttgccagcagccgcgaggcgctgcagggctggggagcgtcgccggcagccggctggccgaaccgcgaaacgaatggatacgcccaagacaggaggcctggggcacaagcgccgcttacgcaagagagcgcatgggacctcggatcgtggacgtcccatacagagcaccaaccgtcgatgagtcggaacgcgatgcttgggagtgccgttggtgcggggcggagtactggaaccaacgaagcgcggaggagcacggccgagcctgcagcaggcgcccgcgcacacggcccgagccagcctgggaacctcctacccagcaccagcgtatacaggagtgggcggatggagcagcctacccaatccaggcgctacccggcgccgttgcgaaccaagcccacgaagagccaacctcacgtggaacaggcgggcgggtataccaggcagcgtgcgacggcgacggacgtgttgagcgacgcaggaggcggccatccaacgcaacgaccactgccacgacgcggagctgggttcgacacagcgatgggtcactcaccgtacgaaccgagcgccgaattaccgacagagagccagcaatcgtacgcgctatcgcccggttcatttcaggatgatgccttggctgccctatcgacactgggtaaggtgccggcattggaggtggcgggaatagtacggcgaggggccactatactgggaaagctcgagccgccgagcgaggagcagacgtacgctcgcctgtaccgtgaggcgcgtgaatacgtgggtgattcgcaggaggtggaggctgaccgcccggtggacagggtgtgggctgaaacctccgagccacgcctgtctcgcacggccatggcgcgggttgaccccgacacgcagtggaagccagacctgtacctggggttgccgtacggccccaacgccgcccaaatcgtggcccagccgttagatgtgcgggaggcgagccactttagcaacctagccccgtggatcataggtgtgctcaacgggacaacgggggcgttcaagggggatgcactgatgctttcggccaaaaccaccccccacgttgacgatgggtggctcggcacacaagctttgaccagacacgacatagacgtcaggctagcagtcgcggatacggcctgcacagtgtccgtgtgggtgggtgtggattatgtggctgggaaaccggtactgcatcacatggctgtggcggggtatcgtgccgcgaagccgccagtgcggcgcctggcactagctctgtgcgaggcgctgacataccatgttgcggtgggtggggttctgccagtgaatgcggtgcataagcgacagtgctccaactacactgatataatgtccgctgaagcgtatagtgacccgccggcgcctaaccaactgggcccgcgtgtggctactaaccctccggagggctgcctgtcccagctggtgcaggtgcaccagtacgacgtgcagactggggttgcaacaggggtcatggcagcgagagacgtgccacctgccgcgttctacttcggcgggacgcccaacggccggaacacagggtacgctatcgcgcgaactgaagtagatggcgtgatgactcacgtgctcacgggcgcagtacacatgaagggacaccccgcgttcctgctctcagggcgtagacgccagccgtgcgtcagggacgggttgtactcgccgggcgctgtgtcgaagctgttcggcgacgggagcggcggcctgtgtatgagggcggaagaggcctcgggagtggaccgcggccttcgctccgtgtggtccgacgccttgtcaaacgctaacaaaactgccgctgtccgctcacgcgacccgctgtcggctgcgaacctcctgatggccatagcaaagcttaacggatggcaagcgaaaccggccgggccgcacagcgtcaccgccgacactgaccaaggccaagtgcgggtgatcgttgagttttggccaacgtctggtccacgttggttggaggtgctgtcattcgacgaggaggtagatcttgggccagagggcgacgatgggcaatgcgatcatccatccgagctggccctcgattattggctgtcgggtttggcaaggtacatgctgaaggacataaccaggaatgggtacctacttaagggctgcggcaagtatgcacgcaacgagctgtctccgttccaagcccaacacacagcggcgccccaagcggcgtgcgatgtggtggtgagcgcgtggcgggtggagacccgggtcggctcagcgaaagcatcttacatgtacaacttgggggtggctgtctgtgcagccggagccgtggtggctacgtcgaacctggttgatgcgaacgcggagcagcggcgcagtgcagtggtgggcaacgggaacgtgctggcggcgtatgataggcgcacacgacccgacgccgcacacgctgacctgctagacgtgctgaagggcctatcaagattggccatggcagggtcagtgcgcgtgtacagtgcaacccaggccggccacgatcaggcagacgtgaacgtgctgagaagcgatggcaagagtgggccgagcgcaagaattatggagatagggacgttagtagagcggcgaggcagcgcagcagatgccgggatggatgcggcaggggctgcggtatcgagcggcgtggcggcagacgacggccagcccgtgagactggcggggactgatgagctgtggtcgacgttgcggcggttagtgaggtgagcgcaggaggccagctcgcctcccccagctggcaaccgaagatatgcggtgataaccgcttaagcgcggtcgccagcatgaacttcttacgtgtcaacgtaagtacc

SEQ ID NO: 3 - DNA, 2875 bp, Mycovirus MoCV3
  CDS (148)..(2499)
gcaaaaaagagaataaagctttctcctttttgcaacgatcgtgcgactccaaactgctgtgtcgataacaacctttgacgcttgacaagccgcgctaattaacaccccacacaacacaacactccagccaactttacttagtgcaacatgggcttgacacttgacccagcgcaccgctggcgctccaccgacttggcgttcgcgcctgtacgggagatcagcgtgcaaaccgccgttcgtggaagggacgacagcgaagcggcggggacagggttcagcggggacaaggagactgaggcatggtacaacgggtcggctcgagtgccgatagacgacatgtgtcttaccgccgggatgcgggcaggtgtcttgaaactggcagttgagataggctccgcaaagccaagctcagcggatgaggcggtagtgcagctgaggtcagtgccttactcgtacgtgggtaagccactgacagtgtcgcttagccacgcggggcggcatttcgtggcgagaccggcgttgaacgagacagtggctatggccatgtacgaggcgcctactgctgacaagtgggtcgccgcaaccaatttcaagctgcccaggaccgtcgccgcgccgggtgctgcgccacaagtgcctggcttgccgaacgggggaggaggcgccaatctaggcttgccgaacaattttgatgctgtgcgacgagtgctggtagaatgcgctcgcggtgacgattacgggtacaggctgtttagcttggctagagtggtgatgcatgcggagacgatgcgtaggtcaggcatatcaccgcgggagacaccggtgatggccgatcagaacatgttcagcataacaactggcgacaccccccatctaacagaggcgcagataaacaactacgcctacgcctacaaccacactgagcaatcgccccagtaccgcgcgttcctaactatgggccttcgaggcgttggtcattatgccatgccggggacaatatactccgacggcgactacccggcggagtgtgccgccaaccatccaatatcattcgttcgtgtgggcgggccgccaccagcgaacgtggctccagacccgccgcactacaccgctgtgttgtctaacccgggcctagccctgtcgtactactgggcgtatgcgtactcaatggggctagggagagtggcgggagcgatcctagcgcaagcgagcttggccccgcatatttggggttcggctgcagtggctccgtacaagaactgcgcgccgaagctggacgcagccgcgtacctactgctgccggaccaggagacagcacacgtcacagctgacagcgcccgcgagttggtcgctaacgccgctgtgctatcggaggcttacctggctggcataggggccacactaatgagcgcgagagatggtgggcatcaagacactgctctgatgatgcgcgcggtcacggagaagttgtctgatccggagactaggcgtggggccatgttgtcgataaccagccgactgtgcccaggcggtgtggggatggagtggttgagtccgttcagctacgacgtcttggatggcacggaacggtgcatacgcgcctggcgcaaccacgggtttttgctggcgctgtatgacacttcacctgtggccgcgctggcaccgctgttttccacaggcgtcgcaatgaacaactcacttctggaccggaagagcgtagtgaccggggctgaatacccgcagttggtggcgtgcgccctcgcgggcagggcagagctagctgggcggtgcgagaagccgtcgcaggcttacctggctgccctagcaggccacagtgcgcggatgagagcgtggacggtggtggtgacggtgctgggagtggtccccccggcatcggacgatgaagacctcgctgacgcgtacgagcaggtggtaagcaggcaagaatcatccagtagcgcaagacctcagagtagtcagagtgacaggagtgttgtgcgcggccacggtgcacaggagcaaacaccgtcgggtgcggcgccgtcgagcccgccgccaatcgccccgctgagaggcatgcgcccggggtcgcgtgcacgtagcagcaaaggctccttatctgtgccgaagggccaattaccggaggtcggggaggagccagcagagcggccgttggatgatagagcggagcccgagccgcctgctgccttaagtccgcccaagctggctctcgacaagccatcgtggggtagctgggcgtcggaggtggcatcagtggaggctaggctcatcggtccgatcagcgggccgaaagggcagattgtcgaacctgagtacagggggatagtcccatcccgcggcacgacaaccgccgcgtccatggcatccgggactgtggtgtcggtgggcaggcgggccaagggtaaggagccagagagagcgcaatcggctagcagtagttctgaacccgccagcggagggtcgcgcggggaggagttgtagcctgcgccgacgaggactagagcggtcaaggtacgcctgttcgagagcgggcgagtggctgcatcacgcaagacgtgaagggcagctgggtcgcaggctggcccgtccggagtgggcgcttggggagaggcagcccagccggtggttttggctagtgcatcggcgacacgtgtccgctgaactggtccagccgtatggtgaggccacatacatgtgccctgccgccgtgtcgggcacgacggcgttggacagggcggtgtgggggttcgagcgcagccgcaggtgatgcgctcagccgccaaccccataattagcgcgcccgcaagttagcgcgcacacatcttgttcgagacgtagggtcccaacaagtacc

SEQ ID NO: 4 - DNA, 2997 bp, Mycovirus MoCV3
  CDS (163)..(2595)
gcaaaaaagagaataaagctttctcctttttgcacaacgcacacgatcgtgaaacacttcaacgttcagcatcgcacgagcagccgttttgatcatacaacttcgaactgcgcgaccgacaggctaggcacgtagttgcagtgtcgtaggtaaataactaaaatggcaatgggcatgacaggcggtcagctagcgtcgaagctgcgtatcgaccaagggttggggttgcctgatgcgttcttgaaacagagtgggcgggccggcgaggctgacagtggcgctgacggcgagtgggccgctttcgggatacactctggggctccccccaacgccgcccacccccacgtggtgcctgccgcactagatgcgtcatcgagcgccgggcgggtagacactttgcgtccactggtgggagacgtggcctacagcctgtttttgaggttaggagagactgactacgacgtgaccaaagatgaagaggccagcccgatggatgtgagccactccgtgatatgctcgtacgctcttgagatcgatgggcgcactgcgctgacgcgcgcggatgtcacgtcgcactgtgctgtgtacccaccgatggccaagagggggagtgccaccccggtgtcggtgacgggcgcgttaacgacgtcacgacgcacgagcgcggccgccgcctgcgaggggctggcaatgcacgcagggtcgggcaataacgcggatgtcagcctgggggtggcccgcgagttcatgtacgacagggcgcggcaccaagagaacgggcttgagtctgtgtttgtgcgtatgtggctggtgcatctcagtgtgttgtcgcggcgacccgtgacacaggtggtggacccagctatgctggctgctcggttcgtgaacatcctagctccagcggagagcgacgccgccagggcaatgcgcggggtcaggataaacgctcgcgggttgagtgacacggcgctggcgctgttggtgctgggatgcagcgacacgtcacaggcggcggggctgcactaccgggcacgacggtacaagttcacgcgctcggcattgtccatgtacggtatgcaggggcggatccgcttggctgtggcgttggacagggcagaggtgaccggtctcgcgatcgtgtccttagcagagagatacggtgcggaggccgcatgcggtgccggcctgcagactgcgctgatgatgtacggcgtgaacgatagcgggcggtacgtgcgactaaagtgcccggaacccgagttacatgatgatgtggccacgacagcaggcatacgagcactgtcggttagaagctactccgatctatcggataaccgcctcctgtcactgtccttatttgtcggtcgggcatggcgccaggcagccgggcacctactgcggtcctccaccatgcagacgaccactgcagacatagacgcagtggtgaacaccctgctgcccagccaaggagcattgataaaggctgtggggtgtgcccatgccagagctatggggtgggtggcgccgttggttgacacggtgtcctacgtggagcagaactaccgcctattgtgggaggagcggggcgtggtgcattgcctggctctcggcttgcgggtgcccaactccgtgctggaggaggcgacggcggtgatagacgttccttatccgcccgccctgtcgccgtccgacgacccgctgtcagccaagccacgcgcgaacagccttgccactcttgggttggtggaatccttgctgtcgtcgtcaggtgagagcttgtgcgggtccgctcgggcgagaggaaggcgccaggccgggctggtggctgtgcccgcgcaggtagtggctctagccgggcaccgggtgcagttcactctactaagtgtcgctagtggcgtggctgtgaaagtggaggagctgccgacacggccattgctggcagagccgctgcagaccggactgcaggctgttgagtacgtgcaggcgccttgggccccggcgcaggatgcaccggaaccggcgccgcctgtggaatttcgagatacgctcgatcaattcctacatgcatacaggccaagcaggtccgtgctggaacaacctagggtagaggtcacaccccgaatgtcgtcagggtcggacgtgcttaacgtgatgctacccgagggcacacctgcctccctgcgagagtctataataggcgggacattaactccagtcagtggcgcaggcgttggcggctgtgcggaggcaatagcgaactccctgcaagcgcagcacggggtgacagtatctgctggtgagatagagaaggcgatgcagggcgacagcggcggtacctcgctgcaggtagaagcaatggctggcgctctggccaagtttggggactaccgactggtgttactagatgaacactctcagggggtgagtttgaaggcgggagccagcgggagcaagccagtaacaatacatcgcaacgggtcatcctataatgcgctggggcgcggcccggggcgagggatacgcgtcaggctgcgtggcggtggcccgcctgcgcgtgaagcacagccgcagctaaggcgcctatcgcgcagcagctgagctgccacttggccgtgtgcgcgtagccgtggctagcggcggtgccggagacgtgagtcggctgattcggtgggcggtgggcgctgatcgtcgtgattgctagcacgcggggctagtgaggtagtgacgcaccccgtattaggtataggccgctgacgggtggtgcactgaccgagtgggtgacggttggggccgccgccggtagggataggtccacgccgcggtggcacacctcttgttgaacatgcagcttcgacaggtaccacaacgcaccccctgcagcgcacgggactccaacgagcaccaccaggtaggtgccggcggggggcgtcgccggcgcgcgcaggcatgcgacgcggtgatacacttcttgttaaacctagggttctaacaagtacc

SEQ ID NO: 5 - DNA, 2861 bp, Mycovirus MoCV3
  CDS (162)..(2159)
gcattctcctttttgcaacacaccaagtgagagccacgcgaccgccttacgctagaccgaccgcgctaaaacagctgtggaggtatccactacgtcaagcaattagacggcgttagactggaacagttgataaccggctcgtcttacttctagcaacagcgatggacgccaagcagtcgggcgccggcgcgcgcgtgtcggaatggtggccagaccggcaagtgatcagccgagaggcgttgcgtcaacgcacgtcccggacgctcggggcgatgggcggcgcggtgggggtgagtgacacgccgcgcagcttcttcggtgggggcgaggtgcccactgagcggctgaggcgcggattgccaagcgaagtggccggtatgttcactgctgagcaactcagcgaaatgctagacatgtcagcggagcagcgcgttaaggcgctggccgacgcatgggagcgtctggccacagccgaagccagcacagggcccacgtcaagtggactgcaggcgccggcacgccgggcccccaggagcgtcagcgactacaacaccacacaatgggttgccgcggcggctagcgacggcatacgagtgacgcccacactgaccgcggctgcgcgcgcggtggcgcccgccttgaacagggcagtggtgccgggcgggtgccaaccccatgcgagttatgggcgcgcgacagtcgctaccgggactggtgacgtagctccgccgtccgctcgtcaaaccgcggtggtgcaggccattggtgagagcgtgcctacggaaccggtggatgccgaggtgaaagaggggacgcctagccccgcggcaccaccccgcctactgtgcggaaccgcgactctactcgcactgccgcaggtttgctgtgggagtgctctggaccacgggtgcgccgcggccccctgcgcggtcggccaggtgtctggggctgcggcggcggttgcgttggcctcgcgcccggtggtggagcccgacgccgatgcgagcattatgggcgagcgttggaaggtgatgtcaacattggcggcacgattgggcccgatagcacagtgccacttcgggtcatgccgcggtgtgtgtgcatgcacccacaggcacccgctagcccgagcactgatggggatcggggtaggcaccgaggaactgcgtggggcgcgcgtacatctaacgaccccgcaagcgtcacggtccgtggcctccgtcctgcttgagaaggtcggcgggggcagccgtgtggagcgtctcgcggttgagttcgtaaaggctgtggacgggccagtgctgaccagcctggtggtggcaagccgcgatggcaaggcggacagcctgttgaccttggttgctgaggcgatggcacaaggatcgctggcggcgccacgcaccatggtgacatttgggaggggcgacggcggtgggccagacttagaccccaataaactatacgacgcgtgcgtgtactcaggcgagcggggccacaagctaggagtactctctcgagcggtgtggttggaaagcgatgcggatgtccactggatgccgcattccaactcgctcaaggttacgctgcagtgctggaatcggcgtggggactgcaagggggtcagcgcggtgataggacctacgacgtggcgcccgcatgtgggcaattcttcctgggagacgttggggtcggatgtctaccgggactggttcggtttagtcgtcgcgcgcaggggcggcggcgacacaactgtgcggcaccaattgagctacaacccaggcatggaggccgaccagcttggcggtctgctggcgcactccggctgcggcgtgtacgtgagtggcgcagacgatgcggcaaccgtggctgcagtgcacccgtcactgcaggttacgtacactcatataacactggggagtccaaccgctagcaagttagtgcaggcggccaccgcactggacgagtttgtgcggcgaggggggcgtatcgcccatgaggctgcggggacagctgacgaagctgtcataaccgctagggacgcgggcggtcgacgacaggggcgctgtaccaaggcgaaggccactgcgcggtcactcggcccgatagacgatggggcgagccaggaccaagcgccgacacgcccgcgtggcggggcgttgtaggcgtctgccgctgagaacagtgcagcgggggcgctacgaccaagttggtggactggcgtactgtggccatggcgtgggggccacatcaacggcgcgcagagcgggtcgtcgcgcgaaggcgatccggggcatacaggcgtgcggagaccgggcgcgatgactggccggcggtttgcccgccgtttcgccggcggcgcagactactggggcaagctgctgtagacatcttggagcggcagggcgcgtcagcagtcgccaccgagttacgcggcgccggaggacccaccggacgcgtgagcgcgcgtgggcggctgcggggcatgcaagcagcgtgaaccaccataccggcgcaggcgggcacggggatacagccggggtcaggctcaataaccgatgggcgatcgccccgatgcctcacaaacggtgcgcgcggcgggggcaggttgctgtgtgtacttctcaagtaaccagtgtgtttgaacatcgctagcgcggttaggcccgcgccccggcggaccacctcttgtttagcatagagcttgaacgagtgtcgcctggtgagccgtggtgaacgtcgcggggattggtgaggtggatgcggggcgctaggtcgtctccccctggtggcccaaggtttgcgacgacggcaaccaacgcgtggtcgtcggtatgaacttcttacgtgatgacgtaagtacc

SEQ ID NO: 6 - protein, 1127 aa, Mycovirus MoCV3
MAGGYDEDVGSLGSGEPVFGASYTNIWHSTLLAAEGSVAHSGPALYAIVLPLSCGKSSLASVLSGYDIDDMVVNSAALHADDEWRTMLDARSKGWAYEDKAAYRLANDLMLRRARRFLRAFEGDDNAPVVYVHTRELATALGLRIIFDGYVEEAAWLGCRRQLESDAVTRDRDLRAYRGQVAANRAHAIRHRQPEPVPYTSHSRLAEAAEAAITRAGLCAGSPRDLADRTKLCGAPPQIMLDLAHSICRDRHRPAWLRAVAAKLLRYRMGEVLPQEALAADNYSEWARVIHATDQHRVAEAPAQSLRGQNWSEVFPYGAGNSRFALVKIGDWIDCTGTSAMGFGYEWFRQMVTRREGTYEQASCMLLMGDVFDYMAPELHPLIQRLPMGSLRLEHYAEIAKEIHRLVRSSVTLLGRRLDAGQLSVCTYWDCLAGRYLGSGDMEKELADRTSEQKPRVWVSRDGTQSADRFAHEFACEVRALLHQTIADGGEQMRTVTDMVASFDTFLEYRKKWVRPGSVTGSPKADIYLEAVSEREGMIAEVADDIAAMGTYVLANVRLNKAATFEFAEFPAIVKRVLADYVPNSFTRYFIKNEIGKPAGRALYPSHLAHYVAGQFALYALMKAQPIPKVRLASERDVAMDEHWMWMQAREFTVGVMLDYDNFNEKHEFADMQLIMRELKGLYRTAGVLSPDLKTMIDWVAEAYDRTVLEYDGELHSFKHGMLSGQAPTSAINNIINGANKRLLIRQVEELTGRVIFQKRTSGGDDVAGETYSLYDAYLAVKCGQQMGLAFKDIKQLLSSDYYEFFRLFVSVKGVHGSLPRALGSICSGQWSNSVKAKFVDPAAKLSSVTDAAFKIARRAGGNATFREKLCATAFKKWASYNEQALVRGFIHGERHSGGLGVPMSDGSVLRIEPIQWPDEERVRLKGLPKDASQVVVEDAVKQATELVGPDSVESAEVVANRLSEQVFKANVAAMEGSRVGQLLGSWEGPRHVRVHEVLRISEADVAATAPTAEEFRAAYAKHKTIIEYYRKAGAKYDALAGVVKPKAREKLARASCNGTPCDYKKLYFWKEHLTMYGCGTYLLTEDTYDAASMLALVVSSELSNEAVSRRLAECAVALKRAGLVSY

SEQ ID NO: 7 - protein, 934 aa, Mycovirus MoCV3
MLMRESRGMEEEVASSREALQGWGASPAAGWPNRETNGYAQDRRPGAQAPLTQESAWDLGSWTSHTEHQPSMSRNAMLGSAVGAGRSTGTNEARRSTAEPAAGARAHGPSQPGNLLPSTSVYRSGRMEQPTQSRRYPAPLRTKPTKSQPHVEQAGGYTRQRATATDVLSDAGGGHPTQRPLPRRGAGFDTAMGHSPYEPSAELPTESQQSYALSPGSFQDDALAALSTLGKVPALEVAGIVRRGATILGKLEPPSEEQTYARLYREAREYVGDSQEVEADRPVDRVWAETSEPRLSRTAMARVDPDTQWKPDLYLGLPYGPNAAQIVAQPLDVREASHFSNLAPWIIGVLNGTTGAFKGDALMLSAKTTPHVDDGWLGTQALTRHDIDVRLAVADTACTVSVWVGVDYVAGKPVLHHMAVAGYRAAKPPVRRLALALCEALTYHVAVGGVLPVNAVHKRQCSNYTDIMSAEAYSDPPAPNQLGPRVATNPPEGCLSQLVQVHQYDVQTGVATGVMAARDVPPAAFYFGGTPNGRNTGYAIARTEVDGVMTHVLTGAVHMKGHPAFLLSGRRRQPCVRDGLYSPGAVSKLFGDGSGGLCMRAEEASGVDRGLRSVWSDALSNANKTAAVRSRDPLSAANLLMAIAKLNGWQAKPAGPHSVTADTDQGQVRVIVEFWPTSGPRWLEVLSFDEEVDLGPEGDDGQCDHPSELALDYWLSGLARYMLKDITRNGYLLKGCGKYARNELSPFQAQHTAAPQAACDVVVSAWRVETRVGSAKASYMYNLGVAVCAAGAVVATSNLVDANAEQRRSAVVGNGNVLAAYDRRTRPDAAHADLLDVLKGLSRLAMAGSVRVYSATQAGHDQADVNVLRSDGKSGPSARIMEIGTLVERRGSAADAGMDAAGAAVSSGVAADDGQPVRLAGTDELWSTLRRLVR

SEQ ID NO: 8 - protein, 784 aa, Mycovirus MoCV3
MGLTLDPAHRWRSTDLAFAPVREISVQTAVRGRDDSEAAGTGFSGDKETEAWYNGSARVPIDDMCLTAGMRAGVLKLAVEIGSAKPSSADEAVVQLRSVPYSYVGKPLTVSLSHAGRHFVARPALNETVAMAMYEAPTADKWVAATNFKLPRTVAAPGAAPQVPGLPNGGGGANLGLPNNFDAVRRVLVECARGDDYGYRLFSLARVVMHAETMRRSGISPRETPVMADQNMFSITTGDTPHLTEAQINNYAYAYNHTEQSPQYRAFLTMGLRGVGHYAMPGTIYSDGDYPAECAANHPISFVRVGGPPPANVAPDPPHYTAVLSNPGLALSYYWAYAYSMGLGRVAGAILAQASLAPHIWGSAAVAPYKNCAPKLDAAAYLLLPDQETAHVTADSARELVANAAVLSEAYLAGIGATLMSARDGGHQDTALMMRAVTEKLSDPETRRGAMLSITSRLCPGGVGMEWLSPFSYDVLDGTERCIRAWRNHGFLLALYDTSPVAALAPLFSTGVAMNNSLLDRKSVVTGAEYPQLVACALAGRAELAGRCEKPSQAYLAALAGHSARMRAWTVVVTVLGVVPPASDDEDLADAYEQVVSRQESSSSARPQSSQSDRSVVRGHGAQEQTPSGAAPSSPPPIAPLRGMRPGSRARSSKGSLSVPKGQLPEVGEEPAERPLDDRAEPEPPAALSPPKLALDKPSWGSWASEVASVEARLIGPISGPKGQIVEPEYRGIVPSRGTTTAASMASGTVVSVGRRAKGKEPERAQSASSSSEPASGGSRGEEL

SEQ ID NO: 9 - protein, 811 aa, Mycovirus MoCV3
MAMGMTGGQLASKLRIDQGLGLPDAFLKQSGRAGEADSGADGEWAAFGIHSGAPPNAAHPHVVPAALDASSSAGRVDTLRPLVGDVAYSLFLRLGETDYDVTKDEEASPMDVSHSVICSYALEIDGRTALTRADVTSHCAVYPPMAKRGSATPVSVTGALTTSRRTSAAAACEGLAMHAGSGNNADVSLGVAREFMYDRARHQENGLESVFVRMWLVHLSVLSRRPVTQVVDPAMLAARFVNILAPAESDAARAMRGVRINARGLSDTALALLVLGCSDTSQAAGLHYRARRYKFTRSALSMYGMQGRIRLAVALDRAEVTGLAIVSLAERYGAEAACGAGLQTALMMYGVNDSGRYVRLKCPEPELHDDVATTAGIRALSVRSYSDLSDNRLLSLSLFVGRAWRQAAGHLLRSSTMQTTTADIDAVVNTLLPSQGALIKAVGCAHARAMGWVAPLVDTVSYVEQNYRLLWEERGVVHCLALGLRVPNSVLEEATAVIDVPYPPALSPSDDPLSAKPRANSLATLGLVESLLSSSGESLCGSARARGRRQAGLVAVPAQVVALAGHRVQFTLLSVASGVAVKVEELPTRPLLAEPLQTGLQAVEYVQAPWAPAQDAPEPAPPVEFRDTLDQFLHAYRPSRSVLEQPRVEVTPRMSSGSDVLNVMLPEGTPASLRESIIGGTLTPVSGAGVGGCAEAIANSLQAQHGVTVSAGEIEKAMQGDSGGTSLQVEAMAGALAKFGDYRLVLLDEHSQGVSLKAGASGSKPVTIHRNGSSYNALGRGPGRGIRVRLRGGGPPAREAQPQLRRLSRSS

SEQ ID NO: 10 - protein, 666 aa, Mycovirus MoCV3
MDAKQSGAGARVSEWWPDRQVISREALRQRTSRTLGAMGGAVGVSDTPRSFFGGGEVPTERLRRGLPSEVAGMFTAEQLSEMLDMSAEQRVKALADAWERLATAEASTGPTSSGLQAPARRAPRSVSDYNTTQWVAAAASDGIRVTPTLTAAARAVAPALNRAVVPGGCQPHASYGRATVATGTGDVAPPSARQTAVVQAIGESVPTEPVDAEVKEGTPSPAAPPRLLCGTATLLALPQVCCGSALDHGCAAAPCAVGQVSGAAAAVALASRPVVEPDADASIMGERWKVMSTLAARLGPIAQCHFGSCRGVCACTHRHPLARALMGIGVGTEELRGARVHLTTPQASRSVASVLLEKVGGGSRVERLAVEFVKAVDGPVLTSLVVASRDGKADSLLTLVAEAMAQGSLAAPRTMVTFGRGDGGGPDLDPNKLYDACVYSGERGHKLGVLSRAVWLESDADVHWMPHSNSLKVTLQCWNRRGDCKGVSAVIGPTTWRPHVGNSSWETLGSDVYRDWFGLVVARRGGGDTTVRHQLSYNPGMEADQLGGLLAHSGCGVYVSGADDAATVAAVHPSLQVTYTHITLGSPTASKLVQAATALDEFVRRGGRIAHEAAGTADEAVITARDAGGRRQGRCTKAKATARSLGPIDDGASQDQAPTRPRGGAL

SEQ ID NO: 11 - DNA, 34 bp, Artificial
  Synthetic DNA
gccccggggcaaaaaagagaataaagctttctcc

SEQ ID NO: 12 - DNA, 34 bp, Artificial
  Synthetic DNA
gttctagaggtacttacacctcacagcgtaagaa

SEQ ID NO: 13 - DNA, 34 bp, Artificial
  Synthetic DNA
gcgagctcgcaaaaaagagaataaagcattccct

SEQ ID NO: 14 - DNA, 34 bp, Artificial
  Synthetic DNA
gtgttaacggtacttacgttgtcacgtaagaagt

SEQ ID NO: 15 - DNA, 34 bp, Artificial
  Synthetic DNA
gcgtcgacgcaaaaaagagaataaagctttctcc

SEQ ID NO: 16 - DNA, 34 bp, Artificial
  Synthetic DNA
gttctagaggtacttgttgggaccctacgtccga

SEQ ID NO: 17 - DNA, 34 bp, Artificial; identical to SEQ ID NO: 15
  Synthetic DNA

SEQ ID NO: 18 - DNA, 34 bp, Artificial
  Synthetic DNA
gttctagaggtacttgttgaagccccatgctcaa

SEQ ID NO: 19 - DNA, 34 bp, Artificial
  Synthetic DNA
gccccggggcaaaaaagagaataaagcattctcc

SEQ ID NO: 20 - DNA, 34 bp, Artificial
  Synthetic DNA
gttctagaggtacttacgtcatcacgtaagaagt

(19) United States
(12) Patent Application Publication
(10) Pub. No.: US 2014/0037586 A1
(43) Pub. Date: Feb. 6, 2014

(54) MYCOVIRUS, PLANT-PATHOGENIC FUNGUS, PLANT DISEASE CONTROL AGENT, METHOD FOR CONTROLLING PLANT DISEASE, AND METHOD FOR ATTENUATING PLANT-PATHOGENIC FUNGUS

(71) Applicants: Hiromitsu Moriyama, Tokyo (JP); Toshiyuki Fukuhara, Tokyo (JP); Tsutomu Arie, Tokyo (JP); Tohru Teraoka, Tokyo (JP)

(72) Inventors: Hiromitsu Moriyama, Tokyo (JP); Toshiyuki Fukuhara, Tokyo (JP); Tsutomu Arie, Tokyo (JP); Tohru Teraoka, Tokyo (JP)

(73) Assignee: NATIONAL UNIVERSITY CORPORATION TOKYO UNIVERSITY OF AGRICULTURE AND TECHNOLOGY, Fuchu-shi, Tokyo (JP)

(21) Appl. No.: 14/001,476

(22) PCT Filed: Feb. 24, 2012
(86) PCT No.: PCT/JP2012/054554

§ 371 Date: Aug. 23, 2013

(30) Foreign Application Priority Data
Feb. 24, 2011 (JP) 2011-038951

(51) Int. Cl. A01N 63/04; A01N 63/00; C12N 7/00

(52) U.S. Cl. CPC A01N 63/04; C12N 7/00; A01N 63/00
USPC 424/93.5; 435/235.1; 435/254.1; 424/93.6; 435/245; 536/23.72; 435/320.1

(57) ABSTRACT

It is intended to exert a higher control effect on plant-pathogenic fungi. The mycovirus of the present invention has 5 types of double-stranded RNAs, wherein 4 types of double-stranded RNAs out of the 5 types of double-stranded RNAs have 81%, 75%, 72%, and 73% or higher homologies to the nucleotide sequences represented by SEQ ID NOs: 1 to 4, respectively. As an example, a novel mycovirus MoCV3 is used.

### TECHNICAL FIELD

The present invention relates to a novel mycovirus that suppresses the infectivity of a plant-pathogenic fungus against a plant. The present invention also relates to a plant-pathogenic fungus infected by the mycovirus, a plant disease control agent comprising the mycovirus, a method for controlling a plant disease using the mycovirus, and a method for attenuating a plant-pathogenic fungus using the mycovirus.

### BACKGROUND ART

Plant diseases are caused by, for example, environmental factors such as weather or soil, infectious factors such as viruses, bacteria, or fungi (filamentous fungi), physiological disorders, or combinations of these factors. Still, a large number of plant diseases are disincentive to the production of food, flowers, flowering trees, timbers, etc. and mostly have a great economic impact.

Of these factors causing plant diseases, fungi are one of the most important causative factors. Approximately 80% of the plant diseases are allegedly caused by the fungi.

For example, rice blast disease is one of the most important plant diseases that occur all over the world. The pathogenic microbe of this disease is a rice blast disease fungus (scientific name: “Magnaporthe oryzae”), which is a mold (filamentous fungus). A temperature around 25 degrees C. is appropriate for the growth, sporulation, or infection of Magnaporthe oryzae, while this fungus prefers a wet environment. Weather factors such as summer-time low temperature, heavy rain, or lack of sunlight therefore cause an outbreak of the fungus, which in turn brings about the poor harvest or reduced quality of rice, dealing a great blow to the economy.

There exist a large number of other plant diseases, such as sheath blight disease, rust disease, powdery mildew, anthrax, stem rot, downy mildew, and gray mold, which are caused by fungi and have a great economic impact. In this regard, attempts are still being made on the development of novel agricultural chemicals, breeding, etc. (for the control of rice blast disease, see, for example, Patent Literatures 1 and 2).

Hereinafter, mycoviruses, which are an item related to the present invention, will be described.

The mycoviruses refer to viruses that infect fungi. Among the mycoviruses, mycoviruses having double-stranded RNA genomes have been reported. The majority of these mycoviruses latently infect host fungi and rarely influence the traits of the hosts.

The mycoviruses having double-stranded RNA genomes are currently classified into five families including Partitiviridae (scientific name), Totiviridae (scientific name), and Chrysoviridae (scientific name). The Partitiviridae viruses have two linear double-stranded RNAs of almost the same size in their virions and have a total gene size of 4 to 6 kbp. The Totiviridae viruses have one linear double-stranded RNA of 4 to 7 kbp in their virions. In addition, a chestnut blight fungus (scientific name: “Cryphonectria parasitica”) has been found to contain an endogenous virus having a double-stranded RNA of 9 to 13 kbp (hypovirus, etc.).

The Chrysoviridae viruses have spherical virus-like particles and have four double-stranded RNA segments. These viruses are known to have regions encoding RNA-dependent RNA polymerase (RdRP), as in the Partitiviridae and Totiviridae viruses. For example, Helminthosporium victoriae 145S virus (Hv145SV), Penicillium chrysogenum virus (PcV), and Agaricus bisporus virus 1 (AbV 1) are known as viruses belonging to the family Chrysoviridae (see, for example, Non Patent Literature 1).

In recent years, methods involving attenuating pathogenic microbes of particular plant diseases with mycoviruses or the like and using the attenuated microbes to control the diseases have been studied, some of which have been put in actual use. There have been disclosed, for example, a method involving attenuating Cryphonectria parasitica using the full-length cDNA of a virus-derived double-stranded RNA that suppresses the toxicity of the fungus and applying the attenuated fungus to the control of chestnut blight (see, for example, Non Patent Literature 1) and a method involving discovering a double-stranded RNA virus that suppresses a violet root rot fungus (scientific name: Helicobasidium mompa) and using an attenuated strain of Helicobasidium mompa endogenously containing the double-stranded RNA to control violet root rot (see, for example, Non Patent Literature 2 and Patent Literature 3).

Alternatively, Patent Literature 4 discloses a mycovirus that infects Magnaporthe oryzae to reduce the infectivity of Magnaporthe oryzae, and an attenuated strain of a plant-pathogenic fungus infected by the mycovirus. Reportedly, use of the mycovirus disclosed in Patent Literature 4 can attenuate a plant-pathogenic fungus and can provide novel means of controlling a plant disease.

### CITATION LIST

### Patent Literature

Patent Literature 1: JP Patent Publication (Kokai) No. 2004-143045 A (2004)
Patent Literature 2: JP Patent Publication (Kokai) No. 2003-250370 A (2003)
Patent Literature 3: JP Patent Publication (Kokai) No. 2001-78752 A (2001)
Patent Literature 4: International Publication No. WO 2009/093409

### Non Patent Literature

Non Patent Literature 1: C. M. Fauquet, Mary Ann Mayo, J. Maniloff, U. Desselberger, L. A. Ball, “Virus Taxonomy: Classification and Nomenclature of Viruses; Eighth Report Of The International Committee On Taxonomy Of Viruses”, Elsevier Academic Press: p. 591-595
Non Patent Literature 2: Gil H. Choi and Donald L. Nuss, “Hypovirulence of chestnut blight fungus conferred by an infectious viral cDNA.” Science. 1992 Aug. 7; 257 (5071): 800-3
Non Patent Literature 3: H. Osaki et al, “Detection of Double-Stranded RNA Virus from a Strain of the Violet Root Rot Fungus Helicobasidium mompa Tanaka” Virus Genes 25: 2, 139-145, 2002

### SUMMARY OF INVENTION

### Technical Problem

Although Patent Literature 4 described above discloses a mycovirus that attenuates a plant-pathogenic fungus, use of the mycovirus or the attenuated strain of a plant-pathogenic fungus infected by the mycovirus fails to produce a sufficient control rate. Thus, the achievement of a higher control rate has been demanded. Accordingly, an object of the present invention is to provide a mycovirus having a higher control effect on a plant-pathogenic fungus, a plant-pathogenic fungus infected by the mycovirus, a plant disease control agent comprising the mycovirus and/or the plant-pathogenic fungus, a method for controlling a plant disease using the mycovirus and/or the plant-pathogenic fungus, and a method for attenuating a plant-pathogenic fungus.

### Solution to Problem

The present invention has attained the object and encompasses the followings:

(1) A mycovirus having 5 types of double-stranded RNAs, wherein 4 types of double-stranded RNAs out of the 5 types of double-stranded RNAs have 81%, 75%, 72%, and 73% or higher homologies to the nucleotide sequences represented by SEQ ID NOs: 1 to 4, respectively.

(2) The mycovirus according to (1), wherein the 5 types of double-stranded RNAs are polynucleotides comprising the nucleotide sequences represented by SEQ ID NOs: 1 to 5, respectively.

(3) A plant-pathogenic fungus which is a host infected by a mycovirus according to (1) or (2).

(4) The plant-pathogenic fungus according to (3), wherein the host is Magnaporthe oryzae.

(5) A plant disease control agent comprising a mycovirus according to (1) or (2) and/or a plant-pathogenic fungus according to (3) or (4).

(6) A method for controlling a plant-pathogenic fungus, comprising a step of contacting a plant disease control agent according to (5) with a plant.

(7) A method for attenuating a plant-pathogenic fungus, comprising the step of allowing a mycovirus according to (1) or (2) to infect the plant-pathogenic fungus.

(8) The method for attenuating a plant-pathogenic fungus according to (7), wherein the plant-pathogenic fungus is Magnaporthe oryzae.

The present specification encompasses the contents described in the specification and/or drawings of Japanese Patent Application No. 2011-38951 on which the priority of the present application is based.

### Advantageous Effects of Invention

The mycovirus according to the present invention is superior in control rate against a plant-pathogenic fungus to conventional mycoviruses. Thus, use of the mycovirus according to the present invention and a plant-pathogenic fungus infected by the mycovirus can control a plant disease with better efficiency than ever.

### BRIEF DESCRIPTION OF DRAWINGS

FIG. 1 is a photograph showing results of observing the colony of a Magnaporthe oryzae S-0412-II 2a strain or a Magnaporthe oryzae S-0412-II 1a strain in a PDA medium.

FIG. 2 is a photograph showing results of observing the colony of a virus-cured strain of the Magnaporthe oryzae S-0412-II 2a strain or the Magnaporthe oryzae S-0412-II 1a strain in a PDA medium.

FIG. 3(A) is a characteristic diagram showing the amounts of hyphae produced by the Magnaporthe oryzae S-0412-II 2a strain and its virus-cured strain. FIG. 3(B) is a characteristic diagram showing the amounts of hyphae produced by the Magnaporthe oryzae S-0412-II 1a strain and its virus-cured strain.

FIG. 4 is a graph showing the number of lesions resulting from the spray inoculation of the Magnaporthe oryzae S-0412-II 2a strain.

FIG. 5 is an electron microscopic photograph of MoCV3 particles.

FIG. 6 is a diagram showing the constitution of a plasmid obtained by the PCR amplification of a full-length MoCV3 cDNA clone dsRNA1, dsRNA2, dsRNA3, dsRNA4, or dsRNA5 followed by subcloning into pUC19.

FIG. 7 is a diagram showing the constitution of pRSA313, pRSA314, or pRSA315 used for constructing a shuttle vector.

FIG. 8 is a diagram showing the constitution of pRSA316 or pRSA317 used for constructing a shuttle vector.

FIG. 9(a) is a diagram showing results of performing Western blotting using anti-MoCV3 antiserum for a fraction obtained by the fractionation of reconstructed MoCV3 through sucrose concentration-gradient centrifugation. FIG. 9(b) is a diagram showing results of observing MoCV3 virus-like particles under electron microscope.

### DESCRIPTION OF EMBODIMENTS

Hereinafter, the present invention will be described in detail. The mycovirus according to the present invention has 5 types of double-stranded RNAs. Of these 5 types of double-stranded RNAs, 4 types of double-stranded RNAs comprise nucleotide sequences having 81%, 75%, 72%, and 73% or higher homologies to the nucleotide sequences represented by SEQ ID NOs: 1 to 4, respectively. The mycovirus according to the present invention has the function of infecting a rice blast disease fungus (scientific name: “Magnaporthe oryzae”) and attenuating a plant-pathogenic fungus such as Magnaporthe oryzae.

As an example, the mycovirus according to the present invention has 5 types of double-stranded RNAs comprising the nucleotide sequences represented by SEQ ID NOs: 1 to 5. In this context, all of the nucleotide sequences of SEQ ID NOs: 1 to 5 in the Sequence Listing are described as DNA sequences. All of these sequences also encompass RNA sequences (thymine is replaced with uracil).

The mycovirus according to the present invention can be preserved in an endogenous form in Magnaporthe oryzae. One example of the mycovirus according to the present invention can include Magnaporthe oryzae chrysovirus 3 (MoCV3) identified and designated by the present inventors. This MoCV3 has 5 types of double-stranded RNAs consisting of the nucleotide sequences represented by SEQ ID NOs: 1 to 5 and is preserved in an endogenous form in Magnaporthe oryzae.

A Magnaporthe oryzae S-0412-II 2a strain endogenously containing this MoCV3 has been demonstrated to fall under the refusal of deposition in National Institute of Technology and Evaluation (NITE), Patent Microorganisms Depositary on the ground of endogenously containing a virus. This strain is also being stored in Laboratory of Plant Pathology, Tokyo University of Agriculture and Technology and can be furnished to third parties on the condition of compliance with laws and regulations. In addition, this strain is in the process of deposition with American Type Culture Collection.

The place where this fungal strain has been originated is Vietnam. Of the double-stranded RNAs, the nucleotide sequence represented by SEQ ID NO: 1 contains a region encoding the conserved motif of RNA-dependent RNA polymerase (RdRP), and this region has a nucleotide sequence homology to Chrysoviridae viruses. Thus, this mycovirus is presumably a novel viral species classified into the family Chrysoviridae.

This MoCV3 is very similar to MoCV1 disclosed in International Publication No. WO 2009/093409, but differs in its excellent ability to attenuate Magnaporthe oryzae, compared with MoCV1. The 4 types of double-stranded RNAs represented by SEQ ID NOs: 1 to 4 out of the 5 types of double-stranded RNAs in MoCV3 exhibited 80.5%, 74.5%, 71.3%, and 72.5% homologies, respectively, to 4 types of double-stranded RNAs in MoCV1. Specifically, a mycovirus comprising 4 types of double-stranded RNAs comprising nucleotide sequences having 81%, 75%, 72%, and 73% or higher homologies to the 4 types of double-stranded RNAs represented by SEQ ID NOs: 1 to 4, respectively, is incorporated as a novel mycovirus in the technical scope of the present invention.

Alternatively, the mycovirus according to the present invention may be a mycovirus that has 5 types of double-stranded RNAs having, for example, 85% or higher, preferably 90% or higher, more preferably 95% or higher, most preferably 97% or higher homologies, to the nucleotide sequences represented by SEQ ID NOs: 1 to 5, respectively, and can attenuate a plant-pathogenic fungus such as Magnaporthe oryzae. In this context, the “homology” refers to being completely identical between nucleotide sequences. “Similarity” refers to being completely identical between nucleotide sequences as a result of replacing adenine with guanine (or vice versa) or thymine (uracil) with cytosine (or vice versa).

The mycovirus according to the present invention is not limited to this MoCV3 and also encompasses a mycovirus artificially or naturally mutated from MoCV3. Specifically, the mycovirus according to the present invention may be a variant that comprises a nucleotide sequence derived from any of the nucleotide sequences represented by SEQ ID NOs: 1 to 5 by the substitution, deletion, addition, or insertion of one or several (e.g., 2 to 100, preferably 2 to 50, more preferably 2 to 25, most preferably 2 to 10) bases and can attenuate a plant-pathogenic fungus such as Magnaporthe oryzae.

The nucleotide sequences represented by SEQ ID NOs: 1 to 5 contain coding regions of their respective proteins. The amino acid sequences of putative proteins encoded by the coding regions contained in the nucleotide sequences of SEQ ID NOs: 1 to 5 are shown in SEQ ID NOs: 6 to 10, respectively.

The mycovirus according to the present invention has the effect of suppressing,' for example, the growth of a plant-pathogenic fungus. Thus, for example, a predetermined plant-pathogenic fungus can be allowed to endogenously contain this mycovirus through its infection or the like to thereby prepare an attenuated strain of the plant-pathogenic fungus.

For example, a plant disease control agent containing the mycovirus according to the present invention and/or the attenuated strain of the plant-pathogenic fungus thus prepared may be added (e.g., distributed or applied) to a plant (rice, etc.) to thereby control its plant disease. In addition, the mycovirus according to the present invention has features as shown below. Mycoviruses have heretofore been considered to be transmitted vertically from the cells of host fungi to cells of other fungi through hyphal fusion and not to exist outside the cells of the host fungi at any stage of their life cycles. By contrast, the study of the present inventors demonstrated that the mycovirus according to the present invention can exist even extracellularly.

Thus, the mycovirus according to the present invention can infect a host fungus from outside its cell in a manner independent of hyphal fusion. The mycovirus according to the present invention can therefore cause transmission even between a wide range of fungal species or fungal strains differing in mating type and can infect a host fungus with high efficiency. This is likely to achieve simple and highly efficient attenuation of a plant-pathogenic fungus or control of a plant disease.

Since the mycovirus according to the present invention can exist even extracellularly, for example, the host fungus is cultured in a liquid medium and the mycovirus can be recovered from a culture supernatant thereof to thereby produce the mycovirus simply and in a relatively large amount.

For example, the mycovirus according to the present invention endogenously contained in a predetermined strain of Magnaporthe oryzae can be separated and recovered from the Magnaporthe oryzae strain by an approach known in the art. In this context, the Magnaporthe oryzae strain endogenously containing the mycovirus according to the present invention can be cultured in the same medium under the same culture conditions as in usual Magnaporthe oryzae.

<Gene, Nucleic Acid, Protein, Etc. According to the Present Invention>

The present inventors have conducted sequencing analysis on one mycovirus according to the present invention to obtain its full-length nucleotide sequence (SEQ ID NOs: 1 to 5). Thus, the present invention encompasses all of genes of the mycovirus, nucleic acids having their nucleotide sequences or a portion thereof, proteins encoded by these nucleotide sequences, etc.

The present invention also encompasses all of nucleic acids havinSg the whole or a portion of these nucleotide sequences. The nucleic acids may be double-stranded or single-stranded and encompass all of DNAs, cDNAs, RNAs, etc.

The present invention also encompasses, for example, cDNAs having all or any of the sequences of SEQ ID NOs: 1 to 5, the sequences of particular sites having predetermined functions in (any of) the sequences of SEQ ID NOs: 1 to 5, or nucleotide sequences equivalent thereto, and recombinant vectors (plasmids, viruses, etc.) with incorporated nucleotide sequences equivalent thereto.

The results of sequencing analysis demonstrated that: a partial site of the sequence of SEQ ID NO: 1 contains the conserved motif of RNA-dependent RNA polymerase (RdRP); and a partial site of the sequence of SEQ ID NO: 4 has a homology to the double-stranded RNA fragment of a La France disease virus. Thus, for example, nucleic acids or recombinant vectors having at least these partial sites of the sequences as particular sites having predetermined functions may be prepared and used according to the purpose or use.

The nucleic acids (or genes) according to the present invention encompass a wide range of nucleic acids having homologies to the nucleotide sequences described above, for example, nucleic acids that hybridize under stringent conditions to nucleic acids consisting of nucleotide sequences complementary to the nucleotide sequences described above and have the effect of suppressing Magnaporthe oryzae. In this context, the stringent conditions can be determined by a technique known in the art with reference to, for example, the Tm values of double-stranded nucleic acids.

The present invention also encompasses all proteins encoded by the mycovirus genes and nucleic acids described above. The amino acid sequences of the proteins according to the present invention are shown in SEQ ID NOs: 6 to 10.

SEQ ID NO: 6 represents an amino acid sequence for an open reading frame in the nucleotide sequence described in SEQ ID NO: 1. SEQ ID NO: 7 represents an amino acid sequence for an open reading frame in the nucleotide sequence described in SEQ ID NO: 2. SEQ ID NO: 8 represents an amino acid sequence for an open reading frame in the nucleotide sequence described in SEQ ID NO: 3. SEQ ID NO: 9 represents an amino acid sequence for an open reading frame in the nucleotide sequence described in SEQ ID NO: 4. SEQ ID NO: 10 represents an amino acid sequence for an open reading frame in the nucleotide sequence described in SEQ ID NO: 5.

The proteins according to the present invention encompass all of proteins having any of the amino acid sequences of SEQ ID NOs: 6 to 10 as well as proteins that have homologies thereto and maintain their functions.

For example, the nucleic acids described above may be incorporated into recombinant vectors and forcedly expressed by hosts to thereby prepare their proteins in large amounts. Any host known in the art, such as E. coli strains, yeast strains, or cultured cells, may be available. Considering that, for example, the mycovirus infects a fungus and yeast strains are highly proliferative and are relatively conveniently used, the yeast strains may be the optimum hosts. Likewise, any recombinant vector known in the art may be available.

For example, the 5 types of genes can be coexpressed using a plurality of vectors each containing any of the nucleotide sequence fragments of SEQ ID NOs: 1 to 5 to thereby reconstruct the mycovirus. In this case as well, any host known in the art and any recombinant vector known in the art are available. Yeast strains are the optimum hosts because a plurality of vectors can be introduced thereinto simultaneously. In this context, the 5 types of nucleic acid fragments represented by SEQ ID NOs: 1 to 5 can be obtained by RT-PCR with the double-stranded RNAs (dsRNAs) extracted from the mycovirus as templates. Alternatively, the 5 types of nucleic acid fragments represented by SEQ ID NOs: 1 to 5 may be totally synthesized on the basis of their sequence information. In other words, those skilled in the art can produce the mycovirus according to the present invention using the recombinant vectors even if the mycovirus itself and/or the Magnaporthe oryzae S-0412-II 2a strain endogenously containing the mycovirus is not deposited with a depositary institution.

<Attenuated Strain of Plant-Pathogenic Fungus>

The attenuated strain of a plant-pathogenic fungus according to the present invention encompasses all of strains endogenously containing the mycovirus according to the present invention. Specifically, the attenuated strain of a plant-pathogenic fungus according to the present invention encompasses, for example, both of a fungal strain (e.g., Magnaporthe oryzae) already endogenously containing the mycovirus and a strain of a plant-pathogenic fungus infected by the mycovirus.

For example, a conventional method involving allowing the mycovirus to infect a host fungus through hyphal fusion can be used as means of allowing the mycovirus to infect a plant-pathogenic fungus. Alternatively, the mycovirus according to the present invention can exist even extracellularly, as mentioned above, and may therefore be allowed to infect a host fungus, for example, directly from outside its cell.

Examples of this attenuated strain of a plant-pathogenic fungus include the S-0412-II 2a strain described above. This fungal strain is a strain of Magnaporthe oryzae infected by the mycovirus according to the present invention. Thus, the S-0412-II 2a strain is basically similar in morphological properties, cultural properties, sporulation, and physiological and chemotaxonomic properties to Magnaporthe oryzae known in the art except that: the strain grows slower than usual Magnaporthe oryzae; its hyphae grow non-concentrically; the strain exhibits nonuniform pigmentation; the abnormal development of aerial hyphae is seen; and sector formation or lysis is observed.

<Plant Disease Control Agent>

The plant disease control agent according to the present invention encompasses all of control agents containing at least any one of the mycovirus according to the present invention and the attenuated strain of a plant-pathogenic fungus according to the present invention. Alternatively, the plant disease control agent according to the present invention may contain both of the mycovirus and the attenuated strain of a plant-pathogenic fungus and may contain an additional ingredient.

The additional ingredient that may be contained therein includes, for example, predetermined carriers, binders, thickeners, fixing agents, preservatives and fungicides, solvents, stabilizers, antioxidants, UV protective agents, crystal deposition inhibitors, antifoaming agents, physical property-improving agents, and coloring agents. The plant disease control agent according to the present invention may also contain additional agricultural chemical ingredient(s), for example, a miticide, a nematicide, a germicide, an antivirus agent, an attractant , a herbicide, a plant growth regulator, and/or a synergist.

For example, either of a solid carrier or a liquid carrier, or both, can be used as the carriers. Examples of the solid carrier include: animal- or plant-derived powders such as starch, active carbon, soybean flour, wheat flour, wood flour, fish flour, and powdered milk; and mineral powders such as talc, kaolin, bentonite, zeolite, diatomaceous earth, white carbon, clay, alumina, calcium carbonate, potassium chloride, and ammonium sulfate. Examples of the liquid carrier include: water; alcohols such as isopropyl alcohol and ethylene glycol; ketones such as cyclohexanone and methyl ethyl ketone; ethers such as propylene glycol monomethyl ether and diethylene glycol mono-n-butyl ether; aliphatic hydrocarbons such as kerosine and light oil; aromatic hydrocarbons such as xylene, trimethylbenzene, tetramethylbenzene, methylnaphthalene, and solvent naphtha; amides such as N-methyl-2-pyrrolidone; esters such as glycerin ester of fatty acid; and plant oils such as soybean oil and rapeseed oil.

Examples of the binders, the thickeners, and the fixing agents include starch, dextrin, cellulose, methylcellulose, ethylcellulose, carboxymethylcellulose, hydroxyethylcellulose, hydroxypropylcellulose, hydroxypropylmethylcellulose, carboxymethyl starch, pullulan, sodium alginate, ammonium alginate, alginic acid propylene glycol ester, guar gum, locust bean gum, gum arabic, xanthan gum, gelatin, casein, polyvinyl alcohol, polyethylene oxide, polyethylene glycol, ethylene-propylene block polymer, sodium polyacrylate, and polyvinylpyrrolidone.

The dosage form of the control agent of the present invention is not particularly limited. For example, forms such as emulsions, suspensions, dusts, granules, tablets, wettable powders, water-soluble powders, liquid formulations, flowable formulations, water-dispersible granules, aerosols, pastes, oil solutions, or concentrated emulsions can be applied thereto.

<Method for Producing Mycovirus Capable of Suppressing Plant-Pathogenic Fungus>

The method for producing a mycovirus capable of suppressing a plant-pathogenic fungus according to the present invention encompasses all of methods comprising at least the procedures of culturing a mycovirus-containing plant-pathogenic fungus in a liquid medium or the like and recovering the mycovirus from a culture supernatant thereof.

As mentioned above, the mycovirus according to the present invention can exist even outside the cell of a host fungus. Thus, for example, a plant-pathogenic fungus infected by the mycovirus is cultured in a liquid medium or the like, and the fungus body can be separated by centrifugation, followed by separation and recovery of the virus from a culture supernatant thereof to thereby recover the virus simply and in a relatively large amount.

However, the mycovirus according to the present invention is not narrowly limited to the mycovirus obtained by this production method. Specifically, the present invention encompasses, for example, a wide range of mycoviruses obtained by separation and recovery from Magnaporthe oryzae endogenously containing the mycovirus.

The method for producing a mycovirus capable of suppressing a plant-pathogenic fungus according to the present invention also includes a method involving, as mentioned above, allowing a host cell to coexpress the 5 types of genes using a plurality of vectors each containing any of the nucleic acid fragments of SEQ ID NOs: 1 to 5 and recovering the mycovirus thus reconstructed in the host cell. A yeast cell can be used as the host cell. The vectors to which the 5 types of genes are incorporated are not particularly limited as long as the vectors can cause the expression of the incorporated genes in the host cell. Any vector can be used. The nucleic acid fragments to be incorporated to the vectors can be totally synthesized on the basis of the nucleotide sequences represented by SEQ ID NOs: 1 to 5.

<Method for Attenuating Plant-Pathogenic Fungus>

As mentioned above, for example, the mycovirus according to the present invention can be allowed to infect a particular plant-pathogenic fungus to thereby suppress, for example, the growth of the host fungus and attenuate the fungus. The same method as above can be adopted as means of allowing the mycovirus to infect a fungus.

<Method for Controlling Plant Disease>

The method for controlling a plant disease according to the present invention encompasses all of methods comprising at least the step of adding the rice blast disease control agent to a particular plant (rice, etc.).

Examples of means of adding the control agent to a plant include a method involving applying the control agent to the front side or back side of each leaf, a method involving attaching the control agent to the front side or back side of each leaf using a predetermined carrier or the like, and a method involving distributing or supplying the control agent to leaves.

The amount of the control agent applied or distributed can be appropriately selected according to various conditions such as the concentration of the active ingredient, the form of the preparation, the type of the target disease or crop, the degree of damage caused by the disease, the place where the control agent is used, the method for use, the timing of use, and the amount and type of a drug, a fertilizer, or the like used together or in combination with the control agent.

For example, a solution containing conidia of an attenuated strain of Magnaporthe oryzae adjusted to 1×10³ to 1×10¹⁰ conidia/mL may be sprayed or supplied in an amount of 1 to 1,000 mL per leaf, or 1×10³ to 1×10¹⁰ conidia of the attenuated strain of Magnaporthe oryzae per mm² of each leaf may be applied or attached to the front side or back side of the leaf, to thereby suppress the plant disease.

Alternatively, a virus solution containing a moderate dilution (approximately 10 to 100 times the undiluted form) of MoCV3 present in the culture supernatant or a moderate dilution of MoCV3 virion components extracted from the fungus body of the Magnaporthe oryzae S-0412-II 2a strain can be distributed directly to rice leaves to thereby achieve a control effect on Magnaporthe oryzae or a method for controlling Magnaporthe oryzae by a curing effect. As an actual control rate, MoCV3 used as a viral spray produced results of 63.6 or higher control rates, which are better than the control rate 56.8 or higher of MoCV1 disclosed in International Publication No. WO 2009/093409.

The present invention may be applicable to every plant disease mainly caused by a fungus. Examples of the plant disease to which the preset invention is applicable include, but not limited to, the followings:

Examples of the plant diseases in Poaceae plants include rice blast disease (causative fungus “Magnaporthe oryzae”), rice leaf spot (causative fungus “Cochliobolus miyabeanus”), sheath blight disease (causative fungus “Thanatephorus cucumeris”), bakanae disease (causative fungus “Gibberella fujikuroi”), damping off (causative fungi “Fusarium spp.”, “Rhizopus spp.”, “Pythium spp.”, and “Trichoderma viride”), rice false smut (causative fungus “Claviceps virens”), wheat head blight disease (causative fungi “Gibberella zeae”, “Fusarium avenaceum”, “Fusarium culmorum”, and “Monographella nivale”), snow mold (causative fungi “Pythium spp.”, “Typhula spp.”, “Monographella nivalis”, and “Myriosclerotinia borealis”), loose smut (causative fungus “Ustilago nuda”), bunt (causative fungus “Tilletia controversa”), eyespot disease (causative fungus “Pseudocercosporella herpotrichoides”), leaf blotch (causative fungus “Septoria tritici”), glume blotch (causative fungus “Phaeosphaeria nodorum”), and powdery mildew (causative fungus “Blumeria graminis”).

Other examples of the plant diseases include: melanose (causative fungus “Diaporthe citri”), melanose-like blemish (causative fungi “Diaporthe medusaea” and “Alternaria citri”), common scab (causative fungus “Elsinoe fawcettii”), brown rot (causative fungus “Phytophthora citrophthora”), green mold (causative fungus “Penicillium digitatum”), and blue mold (causative fungus “Penicillium italicum”) in citrus; monilia disease (causative fungus “Monilinia mali”), scab (causative fungus “Venturia inaequalis”), Alternaria blotch (causative fungus “Alternaria mali”), melanose (causative fungus “Mycosphaerella pomi”), sooty blotch (causative fungus “Gloeodes pomigena”), flyspeck (causative fungus “Zygophiala jamaicensis”), ring rot (causative fungus “Botryosphaeria berengeriana”), brown spot (causative fungus “Diplocarpon mali”), rust (causative fungus “Gymnosporangium yamadae”), and Valsa canker (causative fungus “Valsa ceratosperma”) in apple; scab (causative fungus “Venturia nashicola”), rust (causative fungus “Gymnosporangium asiaticum”), ring rot (causative fungus “Botryosphaeria berengeriana”), and blight (causative fungus “Phomopsis fukushii”) in Japanese pear; leaf curl (causative fungus “Taphrina deformans”), brown rot (causative fungi “Monilinia fructicola” and “Monilinia fructigena”), scab (causative fungus “Cladosporium carpophilum”), and Phomopsis rot (causative fungus “Phomopsis sp.”) in peach; brown rot (causative fungus “Monilinia fructicola” and “Monilinia fructigena”) and young fruit stem rot (causative fungus “Monilinia kusanoi”) in yellow peach; scab (causative fungus “Cladosporium carpophilum”) in Japanese apricot; anthracnose (causative fungus “Elsinoe ampelina”), ripe rot (causative fungi “Colletotrichum acutatum” and “Glomerella cingulata”), brown spot (causative fungus “Pseudocercospora vitis”), and dead arm (causative fungus “Phomopsis viticola”) in grape; angular leaf spot (causative fungus “Cercospora kaki”) and circular leaf spot (causative fungus “Mycosphaerella nawae”) in persimmon; gray blight (causative fungi “Pestalotiopsis longiseta” and “Pestalotiopsis theae”), brown round spot (causative fungi “Pseudocercospora ocellata” and “Cercospora chaae”), blister blight (causative fungus “Exobasidium vexans”), and net blister blight (causative fungus “Exobasidium reticulatum”) in tea; gummy stem blight (causative fungus “Mycosphaerella melonis”), dead arm (causative fungus “Fusarium oxysporum”), scab (causative fungus “Cladosporium cucumerinum”), and brown spot (causative fungus “Corynespora cassiicola”) in gourd; leaf mold (causative fungus “Fulvia fulva”) and ring rot (causative fungus “Alternaria solani”) in tomato; Phomopsis blight (causative fungus “Phomopsis vexans”) and leaf mold (causative fungus “Mycovellosiella nattrassii”) in eggplant; white rust (causative fungus “Albugo macrospora”) and white spot (causative fungi “Cercosporella brassicae” and “Pseudocercosporella capsellae”) in Brassicaceae vegetables; neck rot (causative fungus “Botrytis allii”) in onion; leaf spot (causative fungus “Mycosphaerella fragariae”) in strawberry; early blight (causative fungus “Alternaria solani”) in potato; Phytophthora rot (causative fungus “Phytophthora sojae”) and purple seed stain (causative fungus “Cercospora kikuchii”) in soybean; Phytophthora rot (causative fungus “Phytophthora vignae”) in adzuki bean; brown spot (causative fungus “Mycosphaerella arachidis”) in peanut; brown spot (causative fungus “Cercospora beticola”) and foliage blight (causative fungus “Thanatephorus cucumeris”) in sugar beet; Curvularia leaf blight (causative fungi “Curvularia spp.”), dollar spot disease (causative fungus “Sclerotinia homoeocarpa”), and Helminthosporium leaf blight (causative fungi “Cochliobolus spp.”) in turf grass; scab (causative fungus “Diplocarpon rosae”) in rose; white rust (causative fungus “Puccinia horiana”) in chrysanthemum; downy mildew (causative fungi “Peronospora spp.”, “Pseudoperonospora spp.”, “Plasmopara spp.”, and “Bremia spp.”), Phytophthora rot (causative fungus “Phytophthora spp.”), powdery mildew (causative fungi “Erysiphe spp.”, “Blumeria spp.”, “Sphaerotheca spp.”, “Podosphaera spp.”, “Phyllactinia spp.”, “Uncinula spp.”, and “Oidiopsis spp.”), rust disease (causative fungi “Puccinia spp.”, “Uromyces spp.”, and “Physopella spp.”), black spot (causative fungi “Alternaria spp.”), gray mold (causative fungus “Botrytis cinerea”), stem rot (causative fungus “Sclerotinia sclerotiorum”), white root rot (causative fungus “Rosellinia necatrix”), violet root rot (causative fungus “Helicobasidium mompa”), and southern blight (causative fungus “Sclerotium rolfsii”) in various crops; and other various soilborne diseases (causative fungi “Fusarium spp.”, “Rhizoctonia spp.”, “Pythium spp.”, “Aphanomyces spp.”, “Phoma spp.”, “Verticillium spp.”, “Plasmodiophora brassicae”, etc.).

### EXAMPLES

Hereinafter, the present invention will be described in more detail with reference to Examples. However, the technical scope of the present invention is not limited by Examples below.

### Example 1

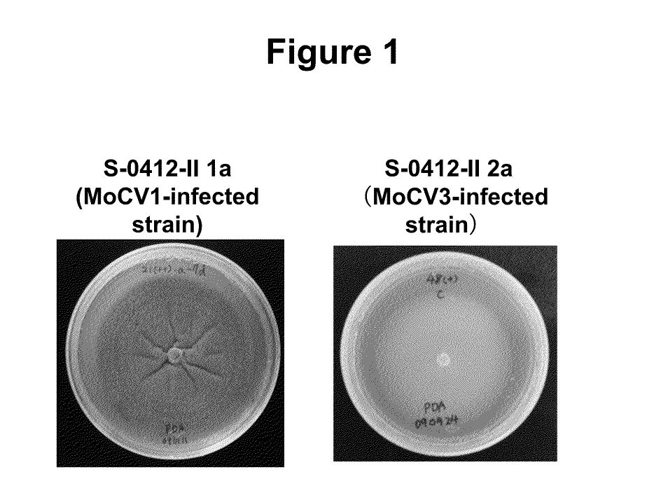

In this Example, a Magnaporthe oryzae S-0412-II 2a strain endogenously containing a novel mycovirus was isolated and identified according to the approach disclosed in International Publication No. WO 2009/093409. Specifically, the Magnaporthe oryzae S-0412-II 2a strain isolated and identified in this Example and a Magnaporthe oryzae S-0412-II 1a strain endogenously containing a mycovirus identified in International Publication No. WO 2009/093409 were separately cultured, and their colonies were observed. A PDA medium was used in the culture of these Magnaporthe oryzae strains. The results are shown in FIG. 1.

As shown in FIG. 1, the Magnaporthe oryzae S-0412-II 2a strain identified in this Example exhibited white albino flora, indicating the observable inhibition of growth. In addition, no conidiation was observed. Unlike the Magnaporthe oryzae S-0412-II 1a strain, the Magnaporthe oryzae S-0412-II 2a strain formed a characteristic colony in such a manner that: pigmentation (melanization) did not occur on a PDA medium; and abnormal and poor growth such as the growth inhibition and wetting of aerial hyphae was exhibited.

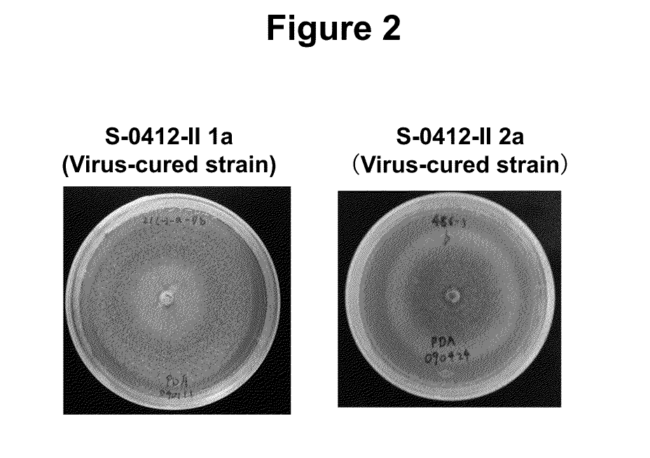

Virus-cured strains of these Magnaporthe oryzae strains S-0412-II 2a and S-0412-II 1a were prepared, and their colonies were similarly observed. The results are shown in FIG. 2. In this context, the method for curing each virus was performed according to the approach disclosed in International Publication No. WO 2009/093409. As shown in FIG. 2, both of the fungal strains formed colonies similar to those of usual Magnaporthe oryzae, as a result of virus cure.

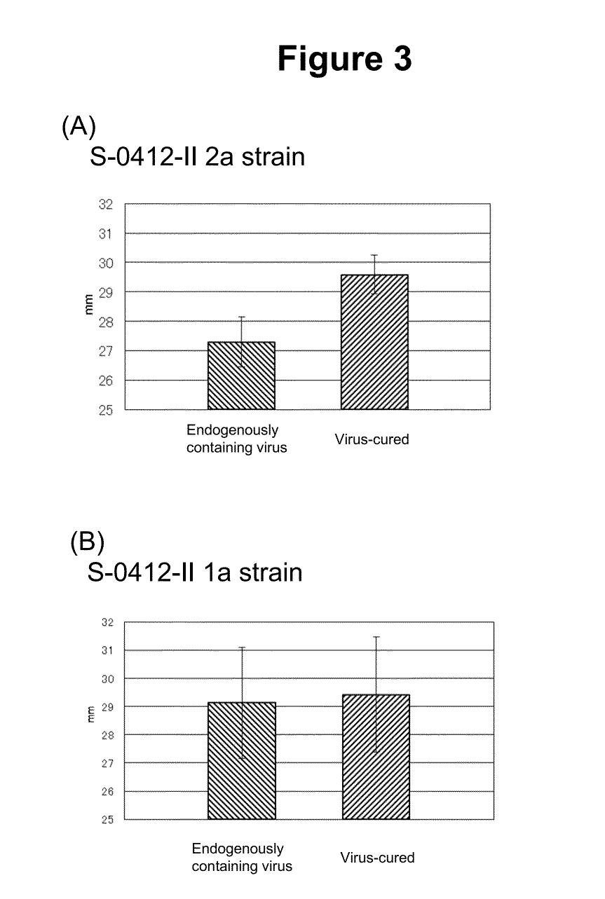

A growth rate was compared between the Magnaporthe oryzae S-0412-II 2a strain and the Magnaporthe oryzae S-0412-II 1a strain and their virus-cured strains. The method for this comparison involved measuring the distance from the center of the medium to the tip of a hypha. In this context, growth conditions for all the fungal strains were set to a cycle involving a light period of 12 hours and a dark period of 12 hours and a culture period of 9 days using a PDA medium. The results are shown in FIGS. 3A and 3B. FIG. 3A shows the measurement results about the Magnaporthe oryzae S-0412-II 2a strain and its virus-cured strain. FIG. 3B shows the measurement results about the Magnaporthe oryzae S-0412-II la strain and its virus-cured strain. As shown in FIGS. 3A and 3B, the Magnaporthe oryzae S-0412-II 1a strain did not exhibit a significant difference in growth rate even after virus cure. By contrast, the Magnaporthe oryzae S-0412-II 2a strain exhibited a significant difference in growth rate as a result of virus cure, demonstrating that the virus-infected strain had a reduced growth rate.

These results of FIGS. 1 and 2 clearly showed that the growth of both the fungal strains was suppressed owing to their endogenous mycoviruses. As shown in FIG. 3, the mycovirus endogenously contained in the Magnaporthe oryzae S-0412-II 2a strain in this Example was shown to have the more potent ability to suppress the growth of Magnaporthe oryzae, compared with the mycovirus (MoCV1) endogenously contained in the Magnaporthe oryzae S-0412-II 1a strain.

The mycovirus endogenously contained in the Magnaporthe oryzae S-0412-II 2a strain isolated and identified in this Example was designated as MoCV3. It was concluded that this MoCV3 was a strong growth-suppressing factor (attenuating factor) against Magnaporthe oryzae.

### Example 2

In this Example, MoCV3 endogenously contained in the Magnaporthe oryzae S-0412-II 2a strain isolated and identified in Example 1 was examined for its existence outside the fungus body.

Of fungal strains with which the bands of 5 segments of endogenous double-stranded RNAs were detected at 2.8 to 3.6 kb in Example 1, 3 strains were separately transplanted and cultured in a liquid medium. Then, each culture solution was centrifuged, and a culture supernatant thereof was recovered. Next, the obtained culture supernatant was electrophoresed on 1% agarose gel at 20 V for 18 hours and stained with ethidium bromide.

As a result, double-stranded RNA bands were also detected, at the same positions as in the endogenous double-stranded RNAs, in the culture supernatants of these fungal strains. This result shows that the mycovirus according to the present invention exists not only within the fungus body of Magnaporthe oryzae but outside the fungus body.

### Example 3

In this Example, the mycovirus MoCV3 existing outside the fungus body was examined for its ability to infect a normal fungal strain (fungal strain carrying no mycovirus).

First, the Magnaporthe oryzae S-0412-II 2a strain was transplanted to a liquid medium and cultured for 4 weeks. Then, the culture solution was centrifuged, and a culture supernatant thereof was recovered. This culture supernatant was electrophoresed on 1% agarose gel by the same procedures as in Example 2. As a result, double-stranded RNA bands were successfully confirmed. The obtained culture supernatant was filter-sterilized through a 0.22-μL filter.

Next, 50 ml of a YG medium was placed in 100-ml Kolben, to which a normal fungal strain (fungal strain carrying no mycovirus) of Magnaporthe oryzae was then inoculated and cultured for 3 days. Then, 500 μL of the obtained culture supernatant was added thereto, and the growth of the fungus body was observed.

As a result, at observation day 3, a fungal strain non-supplemented with the culture supernatant exhibited normal growth with the tips of hyphae growing upright, whereas the hyphae of the fungal strain supplemented with the culture supernatant were entangled with their tips not much growing.

This result suggests that the mycovirus present in the culture supernatant infected the normal fungal strain of Magnaporthe oryzae to suppress its growth. Specifically, the results of this experiment show that the mycovirus existing outside the fungus body has the ability to infect a normal fungal strain.

### Example 4

In this Example, the number of conidia of the Magnaporthe oryzae strain infected by the mycovirus was measured.

The Magnaporthe oryzae S-0412-II 2a strain was transplanted to a YG plate and cultured for 2 weeks. Then, the fungus body was extracted and collected using a cork ball having a diameter of 4 mm. Next, 5% glycerol was placed in a 1.5-ml tube, to which the collected fungus body was added and mixed for 5 minutes for suspension. Then, the number of conidia present in the suspension was counted using a counting chamber.

As a result, the number of conidia was 33×10⁴ conidia/mL for the normal fungal strain (control; fungal strain carrying no mycovirus) of Magnaporthe oryzae, but was 1×10⁴ or less conidia/mL for the Magnaporthe oryzae S-0412-II 2a strain.

This result shows that the mycovirus according to the present invention suppresses the conidiation of a host fungus. This means that MoCV3 can suppress the proliferation of a plant-pathogenic fungus and can also suppress its transmission, the spread of infection, or the like.

### Example 5

In this Example, double-stranded RNAs extracted from the Magnaporthe oryzae S-0412-II 2a strain were sequenced. The extraction and purification of double-stranded RNAs from the Magnaporthe oryzae S-0412-II 2a strain, the synthesis of cDNAs with the obtained double-stranded RNAs as templates, and the subsequent analysis of both terminal sequences by 5′ RACE were performed according to the approach disclosed in International Publication No. WO 2009/093409.

The results clearly showed that MoCV3 contained 5 types of double-stranded RNAs. The nucleotide sequences of these 5 types of double-stranded RNAs are shown in SEQ ID NOs: 1 to 5. Since the double-stranded RNAs were sequenced after reverse transcription into cDNAs, “uracil” is replaced with “thymine” in the Sequence Listing.

As a result of analyzing the obtained nucleotide sequences, the RNA sequence represented by SEQ ID NO: 1 contained the conserved motif of RNA-dependent RNA polymerase (RdRP) that is found in Totiviridae viruses and their related viruses, etc. The RNA sequence represented by SEQ ID NO: 4 contained a region having a homology to the double-stranded RNA fragment L3 of a La France disease virus.

This result shows that the 5 types of double-stranded RNAs according to the present invention offer genetic information on the novel mycovirus.

### Example 6

In this Example, the Magnaporthe oryzae S-0412-II 2a strain was studied by the spray inoculation method for its effectiveness in controlling a plant-pathogenic fungus.

The Magnaporthe oryzae S-0412-II 2a strain was inoculated to an oatmeal medium plate and cultured indoors at 25 degrees C. When conidia were insufficiently formed 15 days after the inoculation, approximately 2 mL of sterilized distilled water was poured to the plate, and the surface of the medium was brushed to remove aerial hyphae. This plate was placed under black light for 3 days to induce conidiation. Approximately 1 mL of sterilized distilled water was poured thereto, and the surface of the medium was brushed again to recover conidia together with aerial hyphae. In this way, a solution containing conidia was obtained. When conidia were sufficiently formed 15 days after the inoculation, approximately 2 mL of sterilized distilled water was poured to the plate, and the surface of the medium was brushed to recover conidia together with aerial hyphae. In this way, a solution containing conidia was obtained. A mycovirus-completely cured strain of Magnaporthe oryzae was prepared as a control by the same procedures as in Example 1. A solution containing conidia was obtained from the fungal strain by the same procedures as above.

Next, each solution containing conidia was filtered through Kimwipe or gauze to adjust the conidia concentration to 2×10⁶ conidia/mL. 0.02% (v/v) Tween 20 was added thereto to prepare a conidium suspension.

Next, the conidium suspension was sprayed evenly over rice seedlings (breed appropriately selected in consideration of a genotype of true resistance to rice blast disease and the race of the fungus; 2 to 3 weeks after seeding) using a nozzle. The plants were left standing for 24 hours in an inoculation chamber set to 26 degrees C. and 100% relative humidity. Then, the pots were transferred to a greenhouse and cultured for 7 days after the spray inoculation while the temperature of the room was kept at 23 to 30 degrees C. Seven days after the spray inoculation, the number of susceptible lesions that became 3 to 4 mm per given area of each leaf was measured.

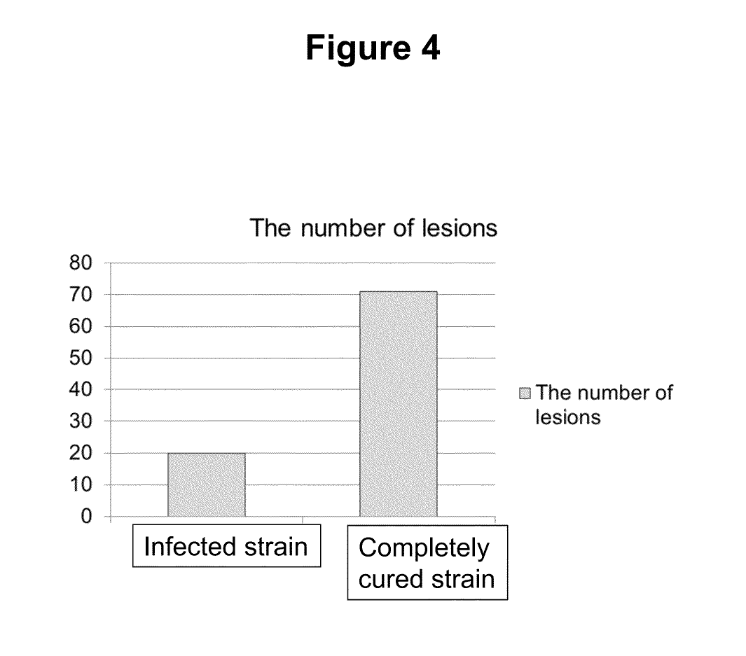

The results are shown in FIG. 4. FIG. 4 is a graph showing the number of lesions in the leaf inoculated with Magnaporthe oryzae. In the diagram, the ordinate denotes the number of lesions in the leaf inoculated with Magnaporthe oryzae. In the diagram, “Mixed infected strain” represents the number of lesions resulting from the spraying of the conidium suspension prepared from Magnaporthe oryzae infected by the mycovirus according to the present invention; and “Completely cured strain” represents the number of lesions (control) resulting from the spraying of the conidium suspension prepared from the mycovirus-completely cured strain of Magnaporthe oryzae.

As shown in FIG. 4, the spraying of the conidium suspension prepared from Magnaporthe oryzae infected by the mycovirus according to the present invention remarkably decreased the number of lesions, compared with the control. This result shows that the Magnaporthe oryzae S-0412-II 2a strain is effective in controlling a plant-pathogenic fungus.

### Example 7

In this Example, the Magnaporthe oryzae S-0412-II 2a strain was studied by the punch inoculation method for its effectiveness in controlling a plant-pathogenic fungus.

A conidium suspension was prepared by the same procedures as in Example 6 and attached to 3% plain agar film. The agar film was cut into approximately 2 mm square to prepare an agar section. The 4th leaf of each rice seedling cultivated in a greenhouse at 23 to 30 degrees C. was punched using a punch for inoculation to form a wet wound. The agar section was placed on the wounded portion. The plant was left standing for 24 hours in an inoculation chamber set to 26 degrees C. and 100% relative humidity. Then, the pot was transferred to a greenhouse and cultured for 14 days after the inoculation while the temperature of the room was kept at 23 to 30 degrees C. 14 days after the inoculation, the size of each lesion was measured.

As a result, the plant inoculated with the conidium suspension prepared from the Magnaporthe oryzae S-0412-II 2a strain had a remarkably small lesion, compared with the control. This result shows that the strain of the present invention is effective in controlling a plant-pathogenic fungus, as in Example 6.

### Example 8

In this Example, MoCV3 particles existing outside the fungus body were identified using an electron microscope. Prior thereto, MoCV3 virions were analyzed for their biochemical properties in the same way as the disclosed approach. As a result, the main component (coat protein) of a virus protein was confirmed by SDS-PAGE to have a molecular weight of approximately 70 kDa. Specifically, the presence of MoCV3 particles was successfully confirmed.

In electron microscopic observation, the Magnaporthe oryzae S-0412-II 2a strain was first cultured in a YG medium (0.5% yeast extract and 2% glucose). Virions were isolated from a culture supernatant thereof. The culture supernatant was centrifuged (10,000×g, 5 min.), and the obtained supernatant was ultracentrifuged (100,000×g, 30 min.) to obtain virion-containing precipitates. The precipitates were dissolved in a 0.05 M phosphate buffer (pH 7.0), then negatively stained with phosphotungstic acid or uranium acetate, and observed under electron microscope (magnification: ×20,000 to 40,000).

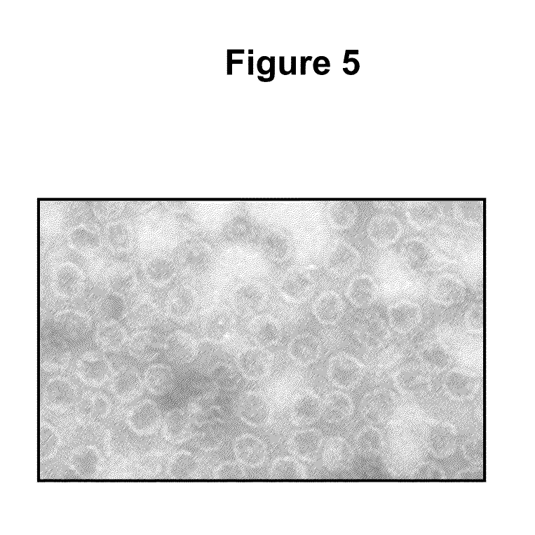

The results are shown in FIG. 5. FIG. 5 is an electron microscopic photograph showing the virions obtained from the culture supernatant of the Magnaporthe oryzae S-0412-II 2a strain. As shown in FIG. 5, the virions of the mycovirus according to the present invention were successfully identified using an electron microscope. These virions were in a regular hexagon-like form of approximately 30 to 40 nm and included in envelope-like structures.

### Example 9

In this Example, isolated MoCV3 was distributed as a spray directly to rice leaves and studied for its control effect and curing effect on Magnaporthe oryzae.

Specifically, MoCV3 present in the culture supernatant was moderately diluted (approximately 10 to 100 times the undiluted form) to prepare a virus solution as a virus spray solution. Next, the diluted virus solution derived from the culture supernatant was sprayed to rice seedlings after a lapse of 2 weeks to 3 weeks from germination. Next, Magnaporthe oryzae was allowed to infect the plants. Then, a control rate in each test section was calculated. The control rate was calculated as Control rate=100−(Damage of treated section/Damage of untreated section)×100 according to Guidelines on Assessment for the Designation of Specified Control Materials (Specified Agricultural Chemicals).

As a result, MoCV3 used as a viral spray produced results of 63.6 or higher control rates. The control rate of MoCV1 disclosed in International Publication No. WO 2009/093409 was calculated in the same way as above and was consequently 56.8. This result clearly showed that MoCV3 endogenously contained in the Magnaporthe oryzae S-0412-11 2a strain was superior in control effect on Magnaporthe oryzae or Magnaporthe oryzae control effect based on its curing effect to the conventionally known mycovirus.

### Example 10

In this Example, the reconstruction of MoCV3 virions in yeast cells was studied. Specifically, in order to reconstruct the MoCV3 virus in the cell of Saccharomyces cerevisiae, full-length cDNA clones were prepared with five dsRNA genomes carried by MoCV3 as templates, and ligated respectively to 5 types of shuttle vectors (see below) having marker genes different from one another. MoCV3 gene products were expressed in Saccharomyces cerevisiae cells to reconstruct MoCV3 virions.

### <Construction of Shuttle Vector for Dene Expression in Yeast>

Backbone vectors pRS313, pRS314, pRS315, and pRS316 were purchased from National BioResource Project (http://yeast.lab.nig.ac.jp/nig/index.html). Backbone vectors pRS317, pRS412, pRS423, pRS424, pRS425, and pRS426 were purchased from ATCC (http://www.atcc.org/).

An alcohol dehydrogenase 1 (ADH1) cassette (ADH1 promoter+MCS +ADH1 terminator) was excised from pAUR123 (manufactured by Takara Bio Inc.) using BamHI and subcloned into pUC19. In order to construct a glyceraldehyde-3-phosphate dehydrogenase (TDH3) cassette, an amplification product containing half a multicloning site (MCS) added to the 3′ end of the 600-bp upstream region of TDH3 and an amplification product containing the remaining half of MCS added to the 5′ end of the 350-bp downstream region of TDH3 were first subcloned by PCR with the genomic DNA of a Saccharomyces cerevisiae strain W303-1A (Mat a ura3 leu2 his3 trp1 ade2 can1) as a template. The obtained two constructs were ligated to prepare a TDH3 cassette having the 600-bp TDH3 upstream region, MCS, and the 350-bp TDH3 downstream region in this order.

MCS was removed from each of the backbone vectors, and the ADH1 cassette or the TDH3 cassette was in turn inserted to the site. The vectors pRS313, pRS314, pRS315, pRS316, and pRS317 having the ADH1 inserts were designated as pRSA313, pRSA314, pRSA315, pRSA316, and pRSA317, respectively. The vectors pRS313, pRS314, pRS315, pRS316, pRS317, pRS423, pRS424, pRS425, and pRS426 having the TDH3 inserts were designated as pRST313, pRST314, pRST315, pRST316, pRST317, pRST423, pRST424, pRST425, and pRST426, respectively.

<Preparation of Shuttle Vector Construct for Expression of MoCV3-Derived Gene (dsRNA1, dsRNA2, dsRNA3, dsRNA4, and dsRNA5)>

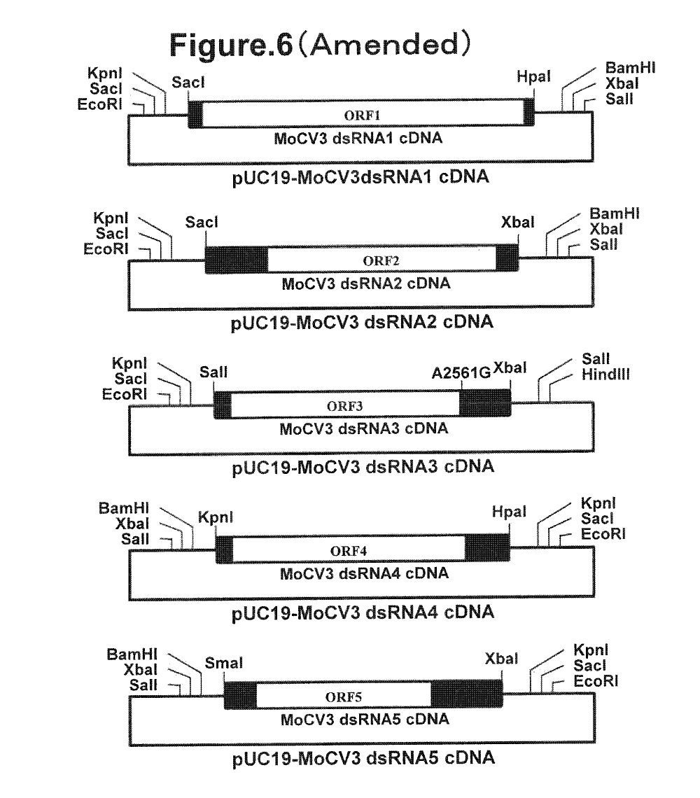

Full-length sequences were amplified by PCR with the full-length MoCV3 cDNA clones dsRNA1, dsRNA2, dsRNA3, dsRNA4, and dsRNA5 as templates. Each obtained DNA fragment was subcloned into pUC19 and then subjected to sequencing analysis to select accurate clones (FIG. 6). The region of interest was excised from each obtained clone using restriction enzymes, then purified, and inserted to the shuttle vectors. Finally, the absence of variation in the inserted sequences and their flanking sequences was confirmed by sequencing.

<Preparation of Full-Length MoCV3 cDNA>
<dsRNA1>

A full-length DNA fragment was obtained by RT-PCR with the dsRNA1 segment separated by native PAGE or purified MoCV3 dsRNA as a template. 20 pmol each of dsRNA1 -specific primers (MoCV3-cDNA-dsRNA1-5end-SmaI and MoCV3-cDNA-dsRNA1-3end-XbaI) was added to the template dsRNA (approximately 50 ng-100 ng) dissolved in distilled water, and the mixture was incubated at 98 degrees C. for 5 minutes and then rapidly cooled on ice for 3 minutes. Then, 40 μl of the reaction system was incubated at 42 degrees C. for 30 minutes and then at 70 degrees C. for 15 minutes under conditions involving 1× first strand synthesis buffer (manufactured by Invitrogen Corp.), 1 mM CH₃HgOH, 10 mM DDT, 1 mM dNTP mix, 40 U RNase OUT Recombinant (manufactured by Invitrogen Corp.), and 200 U Super Script III reverse transcriptase. Then, 5 U RNase H was added thereto. After incubation at 37 degrees C. for 10 minutes and then at 70 degrees C. for 15 minutes, the cDNA was purified and concentrated using QlAquick PCR Purification Kit (manufactured by Qiagen N.V.). This cDNA solution was used as a template in PCR using 2 U Pfu-x (manufactured by Greiner Bio-One International AG) and a buffer included therein, and phosphorylating primers (MoCV3-cDNA-dsRNA1-5end-SmaI: GCC CCG GGG CAA AAA AGA GAA TAA AGC TTT CTC C (SEQ ID NO: 11) and MoCV3-cDNA-dsRNA1-3end-XbaI: GTT CTA GAG GTA CTT ACA CCT CAC AGC GTA AGA A (SEQ ID NO: 12)). The reaction conditions were set to 95 degrees C. for 2 minutes, then 30 cycles each involving 95 degrees C. for 30 seconds, 55 degrees C. for 30 seconds, and 68 degrees C. for 3.5 minutes, followed by 68 degrees C. for 7 minutes. After agarose gel electrophoresis, the cDNA amplification fragment of interest was extracted and purified from the gel and incorporated to a plasmid vector pUC 19 DNA SmaI (manufactured by MBI Fermentas Inc.) using DNA Ligation kit Ver. 2.1 (manufactured by Takara Bio Inc.). The full-length sequence of the inserted fragment in a clone obtained by blue white selection was obtained by sequencing. A clone having a sequence completely identical to that of MoCV3 dsRNA1 was selected.

<dsRNA2>

A clone having a sequence completely identical to that of MoCV13 dsRNA4 was obtained by the same approach as in the preparation of the full-length cDNA of dsRNA1 except that dsRNA2-specific primers (MoCV3-cDNA-dsRNA2-Send-SacI: GCG AGC TCG CAA AAA AGA GAA TAA AGC ATT CCC T (SEQ ID NO: 13) and MoCV3-cDNA-dsRNA2-3end-Hpa1: GTG TTA ACG GTA CTT ACG TTG TCA CGT AAG AAG T (SEQ ID NO: 14)) were used.

<dsRNA3>

A clone having a sequence completely identical to that of MoCV13 dsRNA4 was obtained by the same approach as in the preparation of the full-length cDNA of dsRNA1 except that dsRNA3-specific primers (MoCV3-cDNA-dsRNA3-5end-SacI: GCG TCG ACG CAA AAA AGA GAA TAA AGC TTT CTC C (SEQ ID NO: 15) and MoCV3-cDNA-dsRNA3-3end-Xba1: GTT CTA GAG GTA CTT GTT GGG ACC CTA CGT CCG A (SEQ ID NO: 16)) were used.

<dsRNA4>

A clone having a sequence completely identical to that of MoCV13 dsRNA4 was obtained by the same approach as in the preparation of the full-length cDNA of dsRNA1 except that dsRNA4-specific primers (MoCV3-cDNA-dsRNA4-Send-Sal1: GCG TCG ACG CAA AAA AGA GAA TAA AGC TTT CTC C (SEQ ID NO: 17) and MoCV3-cDNA-dsRNA4-3end-Xba1: GTT CTA GAG GTA CTT GTT GAA GCC CCA TGC TCA A (SEQ ID NO: 18)) were used.

<dsRNA5>

A clone having a sequence completely identical to that of MoCV13 dsRNA5 was obtained by the same approach as in the preparation of the full-length cDNA of dsRNA1 except that dsRNA5-specific primers (MoCV3-cDNA-dsRNA5-Send-Sma1: GCC CCG GGG CAA AAA AGA GAA TAA AGC ATT CTC C (SEQ ID NO: 19) and MoCV3-cDNA-dsRNA5-3end-Xba1: GTT CTA GAG GTA CTT ACG TCA TCA CGT AAG AAG T (SEQ ID NO: 20)) were used.

<Transformation of Yeast with Plasmid DNA>

### <Lithium Acetate Method>

Transformation was performed by the lithium acetate method as follows: first, yeast was transplanted to 5 ml of a liquid medium and precultured at 30 degrees C. for 10 to 16 hours. After the culture, absorbance (OD₆₀₀) was measured, and the yeast was transplanted to 50 ml of a liquid medium at OD₆₀₀=0.1 to 0.2. The yeast was cultured at 100 rpm at 30 degrees C. until OD₆₀₀=0.8. The cells were recovered by centrifugation and then washed by suspension in distilled water. The precipitated cells were suspended in Sol A (0.1 M lithium acetate, 10 mM Tris-HCl pH 7.8, and 1 mM EDTA) and centrifuged. The precipitates were suspended by the addition of Sol A again to prepare a yeast solution. This solution was incubated at 30 degrees C. for 50 minutes. Then, 10 μl of heat-treated ssDNA (1 mg/ml; derived from salmon testis), 50 μl of the yeast solution, 10 μl of each shuttle vector, 20 μl of Sol A, and 750 μl of 50% polyethylene glycerol were added in this order and then incubated at 30 degrees C. for 30 minutes and then at 42 degrees C. for 15 minutes. After centrifugation, distilled water was added to the precipitates. The mixture was applied to an SC plate agar medium (dropout medium was selected according to the selective marker in the shuttle vector) and cultured at 30 degrees C.

### <Spheroplast Method>

Transformation was performed by the spheroplast method as follows: first, yeast cells were cultured and recovered in the same way as the lithium acetate method. The cells were suspended in a 1.2 M sorbitol solution and recovered by centrifugation (2500 rpm, 5 min.). The precipitated cells were suspended again in a 1.2 M sorbitol solution. Zymolyase (20T, 1/10 volume) was added thereto. The cells were incubated at 30 degrees C. until the great majority thereof became spheroplasts. The cells were washed three times with a 1.2 M sorbitol solution and suspended in in an STC solution. 23 μul each plasmid solution, 1 μl of heat-treated ssDNA (1 mg/ml; derived from salmon testis), and 4 ml of a PEG solution were added to 100 μl of this spheroplast suspension and gently mixed. The mixture was left standing for 10 minutes and then centrifuged (1200 rpm, 5 min.) to precipitate the spheroplasts, which were then suspended in 150 μl of an SOS solution. After culture at 30 degrees C. for 1 hour, the suspension was mixed with 5 ml of an SD sorbitol-low melting point agarose solution and applied to an SD sorbitol plate.

### <Electroporation Method>

Transformation was performed by the electroporation method as follows: first, yeast cells were cultured and recovered in the same way as the lithium acetate method. The cells were recovered by centrifugation and then washed by suspension in distilled water. The cells were suspended in 12.5 ml of a LiAC/DTT/TE solution and left standing at room temperature for 1 hour. Then, the cells were precipitated by centrifugation (6,000 rpm, 5 min.). The precipitates were washed twice by suspension in 12.5 ml of ice-cold water. The cells were suspended in 5 ml of a 1 M sorbitol solution and centrifuged (6,000 rpm, 5 min.) to precipitate the cells. The precipitates were suspended in 50 μl of a 1 M sorbitol solution to prepare a cell suspension. 5 μl of each plasmid solution was added to 70 μl of the cell suspension and left standing on ice for 5 minutes. This solution was applied to a 0.2-cm cuvette and subjected to electroporation at 1.5 kV, 25 μFD, and 200 ohms. Immediately thereafter, 1 ml of a 1 M sorbitol solution was added thereto, and the mixture was applied to a 1 M sorbitol plate medium.

<Reconstruction of MoCV3 in Yeast by Introduction of MoCV3 dsRNA1, dsRNA2, dsRNA3, dsRNA4, and dsRNA5>

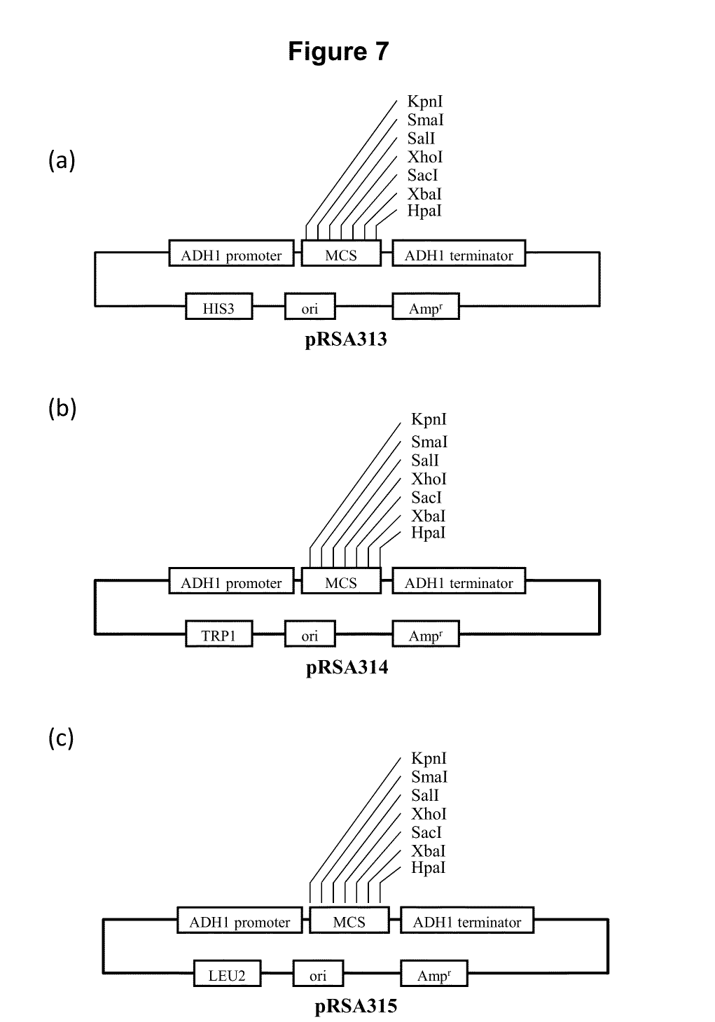

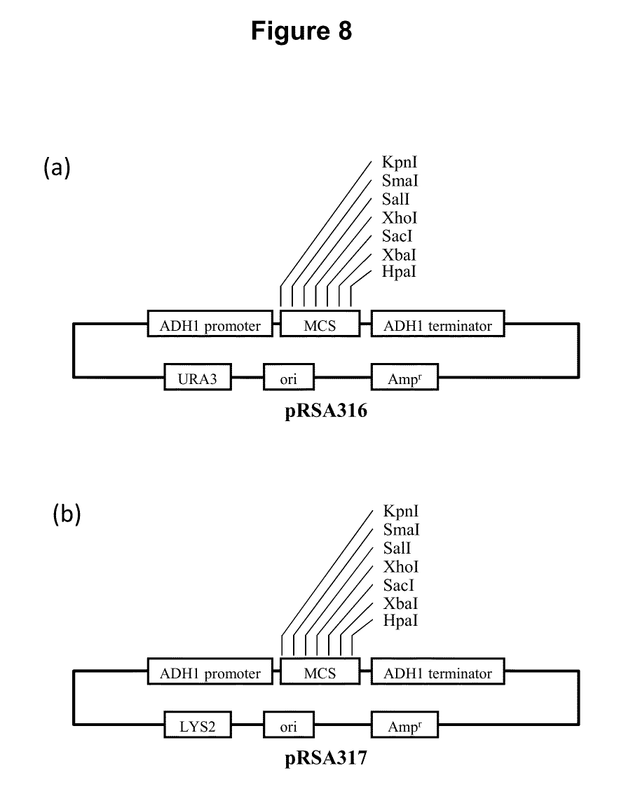

In order to prepare pRSAA313-dsRNA1, pRSA314-dsRNA2, pRSA315-dsRNA3, pRSA316-dsRNA4, and pRSA317-dsRNA5 for expression of MoCV3 dsRNA1 to dsRNA5, shuttle vectors were constructed in which the full-length cDNA clones of dsRNA1 to dsRNA5 prepared by the methods described above were ligated to the restriction enzyme sites of pRSA313, pRSA314, pRSA315, pRSA316, and pRSA317, respectively (FIGS. 7 and 8). For the ligation of the full-length cDNA of dsRNA1 to pRSA313, restriction enzyme sites SmaI and XbaI were used (FIG. 7a). For the ligation of the full-length cDNA of dsRNA2 to pRSA314, restriction enzyme sites Sad and HpaI were used (FIG. 7b). For the ligation of the full-length cDNA of dsRNA3 to pRSA315, restriction enzyme sites SalI and XbaI were used (FIG. 7c). For the ligation of the full-length cDNA of dsRNA4 to pRSA316, restriction enzyme sites Sall and XbaI were used (FIG. 8a). For the ligation of the full-length cDNA of dsRNA5 to pRSA317, restriction enzyme sites SmaI and XbaI were used (FIG. 8b). The dsRNA1 to dsRNA5 gene expression shuttle vectors thus obtained by ligation were introduced to a Saccharomyces cerevisiae YPH499 strain (Mat a ura3 lys2 ade2 leu2 his3 trp1) by the lithium acetate method.

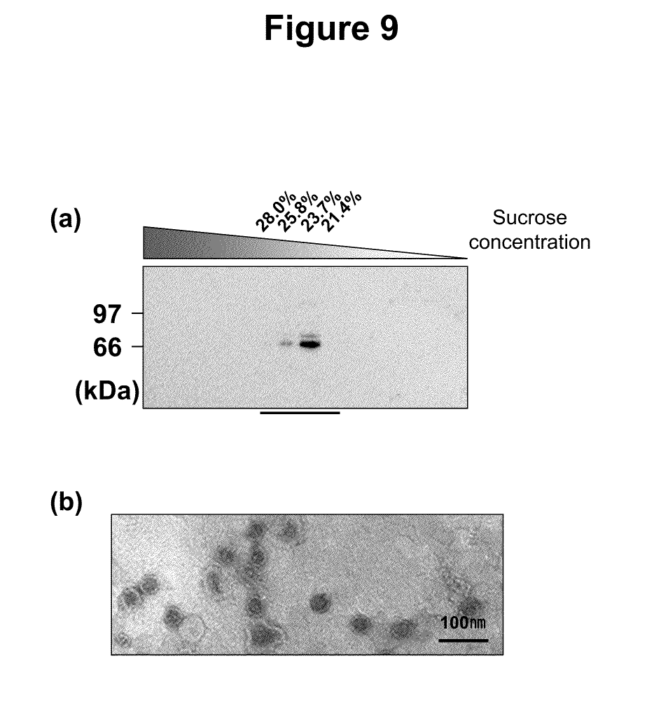

The obtained transformant colony was cultured in a microbial culture apparatus to attempt the purification of virus-like particles. The yeast cells were disrupted using a French press. Then, the homogenates were purified in the same way as the method for purifying MoCV3 virions and fractionated by sucrose concentration-gradient centrifugation. The obtained fraction was subjected to Western blotting using anti-MoCV3 antiserum (FIG. 9a). As a result, a signal that appeared to be derived from the MoCV3 protein was detected around 24% sucrose concentration of the MoCV3 dsRNA1-5-introduced yeast sample. As a result of electron microscopic observation, MoCV3 virus-like particles were observed (FIG. 9b).

All publications, patents, and patent applications cited herein are incorporated herein by reference in their entirety.

1. A mycovirus having 5 types of double-stranded RNAs, wherein 4 types of double-stranded RNAs out of the 5 types of double-stranded RNAs have 85% or higher homologies to the nucleotide sequences represented by SEQ ID NOs: 1 to 4, respectively or encode the amino acid sequences represented by SEQ ID NOs: 6 to 9, respectively.

2. The mycovirus according to claim 1, wherein the 5 types of double-stranded RNAs are polynucleotides comprising the nucleotide sequences represented by SEQ ID NOs: 1 to 5, respectively.

3. A plant-pathogenic fungus which is a host infected by a mycovirus according to claim 1 or 2.

4. The plant-pathogenic fungus according to claim 3, wherein the host is Magnaporthe oryzae.

5. A plant disease control agent comprising a mycovirus according to claim 1 or 2.

6. A method for controlling a plant-pathogenic fungus, comprising a step of contacting a plant disease control agent according to claim 5 with a plant.

7. A method for attenuating a plant-pathogenic fungus, comprising the step of allowing a mycovirus according to claim 1 or 2 to infect the plant-pathogenic fungus.

8. The method for attenuating a plant-pathogenic fungus according to claim 7, wherein the plant-pathogenic fungus is Magnaporthe oryzae.

9. A polynucleotide comprising a nucleotide sequence having 90% or higher homology to the nucleotide sequence of any one of SEQ ID NOs: 1 to 5.

10. An expression vector comprising the polynucleotide according to claim 9.

11. A plant disease control agent comprising the plant-pathogenic fungus according to claim 3.

12. A method for controlling a plant-pathogenic fungus, comprising a step of contacting a plant disease control agent according to claim 11 with a plant.
